(19) United States
(12) Patent Application Publication
(10) Pub. No.: US 2005/0186604 A1
(43) Pub. Date: Aug. 25, 2005

(54) Aberrant Myc/TIP60 interactions as a target for anti-cancer therapeutics

(75) Inventor: Robert Harrod, Rockwall, TX (US)

Correspondence Address: BAKER BOTTS L.L.P.;PATENT DEPARTMENT, 98 SAN JACINTO BLVD., SUITE 1500, AUSTIN, TX, 78701-4039, US

(73) Assignee: Southern Methodist University

(21) Appl. No.: 11/044,865

(22) Filed: Jan. 27, 2005

Related U.S. Application Data
(60) Provisional application No. 60/539,860, filed on Jan. 27, 2004.

(51) Int. Cl. C12Q 1/68; G01N 33/574; A61K 48/00

(52) U.S. Cl. 435/6; 435/7.23; 514/44

(57) ABSTRACT

Human T-cell lymphotropic virus type-1 (HTLV-1) infects and transforms CD4⁺ lymphocytes and causes Adult T-cell Leukemia/Lymphoma (ATLL), an aggressive, often fatal, lymphoproliferative disease. A conserved HTLV-1 3+ regulatory domain, pX, encodes at least five non-structural proteins, including the alternative splice-variant p30^{II}. HTLV-1 p30^{II} may enhance the transforming activity of Myc and transcriptionally activate the human cyclin D2 promoter, dependent upon its conserved Myc-responsive enhancer elements, associated with markedly increased S-phase entry and multi-nucleation. Enhancement of Myc transforming activity by HTLV-1 p30^{II} may be dependent upon the transcriptional coactivators, TRRAP/p434⁶⁻⁸ and TIP60, require TIP60 histone acetyltransferase activity, and strongly correlate with interactions between HTLV-1 p30^{II} and Myc-TIP60 complexes in HTLV-1-infected ATLL patient-derived lymphocytes. Thus, p30^{II} may function as a novel retroviral modulator of Myc-transforming interactions that may prominently contribute to adult T-cell leukemogenesis. Thus, the present invention provides methods and compositions for screening and identifying agents that interfere with transformation.

### RELATED APPLICATION

This application claims priority to U.S. Provisional Patent Application Ser. No. 60/539,860, filed Jan. 27, 2004 and entitled “IDENTIFICATION OF ABERRANT MYC/TIP60 INTERACTIONS AS AN ESSENTIAL MEDIATOR OF NEOPLASTIC TRANSFORMATION: A NOVEL TARGET FOR ANTI-CANCER THERAPEUTICS.”

### TECHNICAL FIELD

The invention relates generally to methods and compositions for the treatment of cancer. The invention also relates to methods and compositions of screening candidate molecules for anticancer activity.

### BACKGROUND OF THE INVENTION

The Myc transcription factor promotes S-phase cell-cycle entry, induces apoptosis or programmed cell-death, and causes neoplastic cellular transformation. Expression of the c-Myc oncogene is deregulated in many solid tumors and hematological malignancies, including Adult T-cell Leukemia/Lymphoma, Diffuse Large-Cell Lymphomas (DLCL), Anaplastic CD30+ Large-Cell Lymphomas, and Burkitt's B-cell Lymphomas (a prominent AIDS-related malignancy). Myc is also deregulated in certain solid tumors containing the Myc gene locus mutations. The transforming viruses, human T-cell lymphotropic virus type-1 (HTLV-1) and Epstein Barr virus (EBV), deregulate Myc functions associated with development of ATLL and Burkitt's lymphomas, respectively.

HTLV-1 infects CD4⁺ T-cells and causes Adult T-cell Leukemia/Lymphoma (ATLL), an aggressive lymphoproliferative disease that is often fatal. HTLV-1-infected leukemic lymphocytes exhibit deregulated cell-cycle progression and characteristic multi-nucleation or polyploidy (evidenced by the appearance of ‘flower-shaped’ or lobulated nuclei). A conserved sequence, known as pX, located within the 3′ terminus of the HTLV-1 genome encodes at least five non-structural regulatory factors, including the viral trans-activator, Tax, and an alternative splice-variant, p30^{II} (or Tax-ORF II, Tof), which possesses a functional trans-activation domain. The pX sequence is generally retained in the majority of ATLL patient isolates -even those containing partially-deleted proviruses, indicative of its importance for pathogenesis.

The viral Tax protein transcriptionally activates numerous lymphoproliferative pathways (NF-κB, CREB/ATF, and p67^{SRF}) and has been shown to inhibit transcription functions associated with the tumor suppressor p53 which likely contributes to a loss of G1/S-phase checkpoint control in HTLV-1-infected T-cells. Many of the pleiotropic effects of Tax upon cellular-signaling may derive from its aberrant recruitment of the transcriptional coactivators, p300/CREB-binding protein (p300/CBP) and p300/CBP-associated factor (P/CAF). Further, Tax interacts with cell-cycle modulators, including D-type cylin-cdk4/6 complexes, retinoblastoma (Rb) protein, and the human mitotic arrest-deficiency-1 (hMAD-1) protein. Although HTLV-1 Tax expression markedly promotes G1/S transition, Tax inhibits Myc-dependent trans-activation and prevents Myc-associated anchorage-independent cell-growth. Since ATLL patient-derived lymphocytes and tumors from HTLV-1 pX transgenic mice are known to possess deregulated Myc functions, other pX-encoded factors may influence Myc to promote cellular transformation by HTLV-1.

Preliminary studies indicate that the HTLV-1 accessory protein p30^{II} markedly increases S-phase cell-cycle progression and induces significant polyploidy. As relatively little is known with respect to the roles of pX-encoded accessory factors (e.g., p30^{II}, p13^{II}, p12^{I}, Rex^{p27}) in HTLV-1-associated pathogenesis, the molecular mechanism by which p30^{II} promotes Myc-dependent S-phase progression and multi-nucleation was sought. While others have proposed that HTLV-1 p30^{II}'s functions are targeted against the viral LTR to repress HTLV-1 gene expression, it remains unclear whether these observations reflect the physiological role of p30^{II}. Nicot et al. (2004, Nat. Med. 10:197-201) and Younis et al. (2004, J. Virol. 78:11077-11083) have shown that p30^{II}, binds and inhibits nuclear export of the doubly-spliced Tax/Rex HTLV-1 mRNA, and it is intriguing that p30^{II}, might perform diverse functions to regulate viral gene expression and promote altered cellular growth—as noted for Tax which drives LTR trans-activation and deregulates host lymphoproliferative-signaling pathways. Robek et al. (1998, J. Virol. 72:4458-4462) have previously demonstrated that p30^{II} is dispensable for immortalization and transformation of human PBMCs by an infectious HTLV-1 molecular clone, ACH.p30^{II}, defective for p30^{II} production. However, the ACH.p30^{II} mutant exhibited an approx 20-50% reduction in transformation-efficiency compared to the wild-type ACH.wt suggesting that p30^{II} is required for the full transforming-potential of HTLV-1.

### SUMMARY OF THE INVENTION

The present invention demonstrates that aberrant interactions between Myc (Accession No. 0907235A) and the histone acetyl transferase Tat interactive protein 60 kD (TIP60) (Accession No. U74667.1) drastically enhance the transforming potential of Myc. In some embodiments of the invention, interaction of Myc and TIP60 may be stabilized through interactions with factors associated with oncogenic viruses such as the HTLV-1 p30^{II} protein (Accession No. AAB23361.1). For example, without being limited to any particular mechanism of action, HTLV-1 p30^{II} may (a) recruit the transcriptional coactivator TIP60 to Myc-containing chromatin remodeling complexes assembled on conserved E-box (CACGAG) enhancer elements within promoters of Myc-responsive genes, (b) transcriptionally activate the human cyclin D2 promoter, (c) increase S-phase cell-cycle progression and polyploidy (multi-nucleation), and (d) markedly induce colony formation in transformation assays using immortalized human fibroblasts.

A trans-dominant negative TIP60MAT mutant which contains an inactivating-deletion within the histone acetyltransferase (HAT) domain, abrogated foci-formation by HTLV-1 p30^{II}/Myc and significantly inhibited trans-activation from the human cyclin D2 promoter. These data indicate that aberrant Myc-TIP60 protein interactions prominently contribute to Myc-dependent neoplastic transformation and transcriptional activity, and further suggest that disruption of Myc-TIP60 complexes and inhibition of Myc-TIP60 complex formation are plausible approaches to impede malignancy in anti-cancer therapies.

Thus, the present invention provides aberrant Myc-TIP60 interactions as a molecular target for the development of anti-cancer therapies. These therapies may impede malignancy or slow tumor progression. The present invention also provides screening methods for the identification of anti-cancer therapeutics that block, inhibit, weaken, or otherwise interfere with interactions between Myc and TIP60.

Without being limited to any particular mode of action, factors encoded by transforming viruses (e.g. EBV, HPV, KSHV) or mutations of Myc may facilitate stabilization of Myc-TIP60 interactions to promote neoplastic cellular transformation.

The present invention also relates to cells that may be used to detect Myc-TIP60 interaction. In some embodiments, these cells comprise:
a first nucleic acid including an expression control sequence having at least one E-box enhancer element and a reporter gene, wherein the expression control sequence is operatively linked to the reporter gene;
a second nucleic acid including an expression control sequence and a nucleotide sequence encoding human T-cell lymphotropic virus type-1 (HTLV-1) p30^{II}, wherein the expression control sequence is operatively linked to the nucleotide sequence encoding HTLV-1 p30^{II}; and
a third nucleic acid including an expression control sequence and a nucleotide sequence encoding human TIP60, wherein the expression control sequence is operatively linked to the nucleotide sequence encoding human TIP60.

In some embodiments, the expression control sequence of the first nucleic acid is selected from the group consisting of a human cyclin D2 promoter and a minimal thymidine kinase promoter. In some embodiments of the invention, the expression control sequence of the second nucleic acid and/or third nucleic acid may be a cytomegalovirus promoter. In some embodiments of the invention, the reporter gene may encode a protein selected from the group consisting of β-galactosidase, β-glucuronidase, autofluorescent proteins, including blue fluorescent protein (BFP) and green fluorescent protein (GFP), glutathione-S-transferase (GST), luciferase, horseradish peroxidase (HRP), and chloramphenicol acetyltransferase (CAT).

The present invention also provides methods of blocking, impeding, or otherwise interfering with the interaction of Myc and TIP60. For example, the invention provides a method of interfering with Myc and TIP60 interaction in a cell, comprising: contacting the cell with a nucleic acid, polypeptide, or organic molecule, wherein the nucleic acid, polypeptide, or organic molecule inhibits Myc-TIP60 interaction. In some embodiments of the invention, the organic molecule is a small molecule drug. The polypeptide may be a protein that comprises a TIP60_{ΔHAT} protein. Similarly, the nucleic acid may be a nucleic acid that encodes a polypeptide comprising a TIP60_{ΔHAT} protein.

The invention further provides screening assays to identify one or more molecules that block, impede, or otherwise interfere with neoplastic transformation. For example, the invention provides a method of identifying a molecule that inhibits neoplastic transformation of a cell, comprising:
contacting a test cell with a test molecule;
measuring the cellular foci formed in the presence of the test molecule; and
comparing the number of foci formed in the presence of the test molecule with the number of foci formed by test cell in the absence of the test molecule,
   wherein formation of fewer foci in the presence of the test molecule than in the absence of the test molecule indicates inhibition of neoplastic transformation, and wherein the test cell comprises:
a first nucleic acid comprising an expression control sequence and a nucleotide sequence encoding the Myc transcription factor, wherein the expression control sequence is operatively linked to the reporter gene;
a second nucleic acid comprising an expression control sequence and a nucleotide sequence encoding human T-cell lymphotropic virus type-1 (HTLV-1) p30^{II}, wherein the expression control sequence is operatively linked to the nucleotide sequence encoding HTLV-1 p30^{II}; and
a third nucleic acid comprising an expression control sequence and a nucleotide sequence encoding human TIP60, wherein the expression control sequence is operatively linked to the nucleotide sequence encoding human TIP60.
   According to the invention, the expression control sequence of the second nucleic acid and/or third nucleic acid may be a cytomegalovirus promoter.

The invention also provides methods for identifying one or more molecules that block, impede, or otherwise interfere with Myc-TIP60 interactions. The invention provides, for example, a method of identifying a molecule that interferes with Myc-TIP60 interaction, comprising:
contacting a test cell with a test molecule wherein the test cell comprises:
   a first nucleic acid comprising an expression control sequence comprising at least one E-box enhancer element and a reporter gene, wherein the expression control sequence is operatively linked to the reporter gene;
   a second nucleic acid comprising an expression control sequence and a nucleotide sequence encoding human T-cell lymphotropic virus type-1 (HTLV-1) p30_{II} wherein the expression control sequence is operatively linked to the nucleotide sequence encoding HTLV-1 p30^{II}; and
   a third nucleic acid comprising an expression control sequence and a nucleotide sequence encoding human TIP60, wherein the expression control sequence is operatively linked to the nucleotide sequence encoding human TIP60;
   detecting the reporter gene expression in the presence of the test molecule; and
   comparing the reporter gene expression in the presence of the test molecule with reporter gene expression in the absence of the test molecule,
      wherein reduced reporter gene expression in the presence of the test molecule relative to reporter gene expression in the absence of the test molecule indicates inhibition of Myc-TIP60 interaction.

According to the invention, the expression control sequence of the first nucleic acid may be selected from the group consisting of a human cyclin D2 promoter and a minimal thymidine kinase promoter. In addition, the expression control sequence of the second nucleic acid and/or third nucleic acid may be a cytomegalovirus promoter. In some embodiments of the invention, the reporter gene may encode a protein selected from the group consisting of β-galactosidase, β-glucuronidase, autofluorescent proteins, including blue fluorescent protein (BFP) and green fluorescent protein (GFP), glutathione-S-transferase (GST), luciferase, horseradish peroxidase (HRP), and chloramphenicol acetyltransferase (CAT).

In some embodiments the invention provides a method of detecting cancer in a test tissue sample, comprising:
detecting Myc-TIP60 complexes in the test tissue sample; and
comparing the Myc-TIP60 complexes in the tissue sample with Myc-TIP60 complexes in a corresponding non-cancerous tissue,
   wherein an elevated level of Myc-TIP60 complexes in the test tissue sample relative to the non-cancerous tissue indicates the presence of cancer. The method of detecting complex formation may be accomplished by any means of detection protein-protein interactions known in the art. For example, detection may be achieved by lysing cells of the test tissue sample, forming a clear extract, and immunoprecipitating Myc-interacting complexes with an anti-HA tag antibody. The test tissue sample may be derived from any source including, without limitation, tissue biopsies.

### BRIEF DESCRIPTION OF THE DRAWINGS

The patent application file contains at least one drawing executed in color. Copies of this patent or patent application publication with color drawing(s) will be provided by the United States Patent and Trademark Office upon request and payment of the necessary fee.

A more complete and thorough understanding of the present embodiments and advantages thereof may be acquired by referring to the following description taken in conjunction with the accompanying drawings, in which like reference numbers indicate like features, and wherein:

FIG. 1A is a diagram of the HTLV-1 proviral genome and its translation products with the the viral transcription factors, Tax and p30^{II}, are in bold;

FIG. 1B illustrates a RasMol structural prediction of the HTLV-1 p30^{II} protein in which sub-domains (4 alpha-helices; 19 beta-sheets) are represented by different colors and Connelly/Richards (1.2 Å) radii are indicated in white;

FIG. 1C, left panel illustrates a plot of DNA content (assayed by 7-AAD) versus DNA synthesis (S-phase; assayed by BrdU content) for Molt-4 lymphocytes that were transfected with an empty CβS vector control (3.0 μg);

FIG. 1C, right panel illustrates a fluorescent activated cell sorting (FACS) analysis of the percentages of apoptotic cells in cultures of CβS-transfected Molt-4 lymphocytes stained with annexin-V-(FITC)/propidium iodide;

FIG. 1D illustrates flow cytometry results using Molt-4 lymphocytes that were transfected with an empty CβS vector control (3.0 μg)

Table 1 shows the relative percentages of CβS-transfected Molt-4 lymphocytes in various stages of the cell-cycle as quantified using aneuploid analysis software (ModFit LT 3.0);

FIG. 1E, left panel illustrates a plot of DNA content (assayed by 7-AAD) versus DNA synthesis (S-phase; assayed by BrdU content) for Molt-4 lymphocytes that were transfected with CMV-HTLV-1 p30^{II} (HA) (3.0 μg);

FIG. 1E, right panel illustrates a fluorescent activated cell sorting (FACS) analysis of the percentages of apoptotic cells in cultures of CMV-HTLV-1 p30^{II}-transfected Molt-4 lymphocytes stained with annexin-V-(FITC)/propidium iodide;

FIG. 1F illustrates flow cytometry results using Molt-4 lymphocytes that were transfected with CMV-HTLV-1 p30^{II} (HA) (3.0 μg);

Table 2 shows the relative percentages of CMV-HTLV-1 p30^{II}-transfected Molt-4 lymphocytes in various stages of the cell-cycle as quantified using aneuploid analysis software (ModFit LT 3.0);

FIG. 2A illustrates the results of immunofluorescence-laser confocal microscopy performed on HTLV-1-infected ATLL patient-derived T-cells (ATL-1, ATL-2, ATL-3) or Jurkat E6.1 lymphocytes as a negative control, using a monoclonal anti-Myc antibody (blue, left panels) or a rabbit polyclonal anti-HTLV-1 p30^{II} antibody (green, left-middle panels), merged images of the Myc and HTLV-1 p30^{II} localization (right-middle panels), and merged images with viewed with phase contrast optics;

FIG. 2B illustrates a three-dimensional Z-stack composite for ATL-3 with three rotational views of merged images (demonstrating nuclear co-localization of HTLV-1 p30^{II} (green)/Myc (blue) in all focal planes) and a graphical representation of relative fluorescence-intensities for HTLV-1 p30^{II}/Myc-specific signals and DAPI nuclear-staining for reference;

FIG. 2C illustrates a co-immunoprecipitations performed using extracts prepared from HTLV-1-infected ATLL patient-derived lymphocytes and anti-Myc or anti-HTLV-1 p30^{II} antibodies;

FIG. 2D, upper panel illustrates the results of co-immunoprecipitation assays of Jurkat E6.1, HuT-102, and MJ[G11] lymphocytes transfected with an empty CβS vector control or CMV-HTLV-1 p30^{II} (HA) using a monoclonal anti-HA tag antibody (CA5, Roche Molecular Biochemicals);

FIG. 2D, lower panel illustrates the results of co-immunoprecipitation assays of Jurkat E6.1 whole-cell extracts using antibodies against known. RNA Polymerase II and TIP48 binding partners (anti-p300; anti-Myc) with an anti-HTLV-1 Tax monoclonal antibody (20) was used as a negative control;

FIG. 2E illustrates the results of co-immunoprecipitation assays of Jurkat E6.1, HuT-102, and MJ[G11] lymphocytes transfected with an empty CβS vector control or CMV-HTLV-1 p30^{II} (HA) using a monoclonal anti-HA tag antibody (CA5, Roche Molecular Biochemicals) in which HTLV-1 p30^{II}-interacting proteins were detected by immunoblotting;

FIG. 3A shows a bar graph with the results of luciferase assays of HeLa cells co-transfected with a human cyclin D2 promoter-luciferase reporter plasmid and increasing amounts of CMV-HTLV-1 p30^{II} (HA) and, in the lower panel, the expression of HTLV-1 p30^{II} (HA), Myc, and Actin in transfected cells;

FIG. 3B shows a bar graph with the results of luciferase assays of HeLa cells co-transfected as in FIG. 3A, lacking conserved Myc-responsive E-box enhancer elements, and increasing amounts of CMV-HTLV-1 p30^{II} (HA);

FIG. 3C shows a bar graph with the results of luciferase assays of 293A Fibroblasts co-transfected as in FIG. 3A with a human cyclin D2 promoter-luciferase reporter plasmid and increasing amounts of CMV-HTLV-1 p30^{II} (HA) and, in the lower panel, the expression of HTLV-1 p30^{II} (HA), Myc, and Actin in transfected cells detected by immunoblotting;

FIG. 3D shows a bar graph with the results of luciferase assays of 293A Fibroblasts co-transfected as in FIG. 3A with a synthetic, E-box-containing minimal tk promoter-luciferase reporter construct (M4-tk-luc) and increasing amounts of CMV-HTLV-1 p30^{II} (HA) (error bars representing standard deviations are provided);

FIG. 4A shows a bar graph with the results of luciferase assays of HeLa cells were co-transfected with a human cyclin D2 promoter-luciferase reporter plasmid (0.5 kg) and CMV-HTLV-1 p30^{II} (HA) (0.15 kg) in the presence of increasing amounts of CMV-wild-type TIP60, CMV-TIP60_{ΔHAT}, or CMV-TIP60_{L497A} and, in the lower panel, the expression of HTLV-1 p30^{II} (HA) and Actin detected by immunoblotting (lower panels);

FIG. 4B shows a bar graph with the results of luciferase assays of HeLa cells co-transfected as in FIG. 4A with a human cyclin D2 promoter-luciferase plasmid and CMV-HTLV-1 p30^{II} (HA) in the presence of increasing amounts of CβS-TRRAPₐₙₜᵢ₋ₛₑₙₛₑ or CβF-TRRAP₁₂₆₁₋₁₅₇₉ and, in the lower panel, the expression of the trans-dominant negative TRRAP₁₂₆₁₁₋₁₅₇₉-(FLAG) mutant, HTLV-1 p30^{II} (HA), Myc, and Actin proteins detected by immunoblotting using an anti-FLAG M2 monoclonal antibody (SIGMA Chemical Corp.), anti-HA (CA5) or anti-Myc monoclonal antibodies, or anti-Actin goat polyclonal antibody (error bars representing standard deviations are provided).;

FIG. 4C illustrates over-expression of the (FLAG)-TIP60 (wild-type) and (FLAG)-TIP60_{ΔHAT} proteins (23) relative to endogenous TIP60 as visualized by immunofluorescence-microscopy using a rabbit polyclonal anti-TIP60 antibody (top panels) and an anti-FLAG M2 monoclonal antibody (bottom panels) with the CβS empty vector transfected as a negative control;

FIG. 5A illustrates the results of chromatin-immunoprecipitation assays performed on uninfected Molt-4 lymphocytes using antibodies that recognize various Myc-interacting factors (TIP60, TRRAP, TIP48, TIP49, hGCN5; upper panel) or acetylated forms of histone H3 (Acetyl-K9, Acetyl-K14; lower panel);

FIG. 5B illustrates the results of chromatin-immunoprecipitation assays performed on HTLV-1-infected MJ[G11] lymphocytes using antibodies that recognize various Myc-interacting factors (TIP60, TRRAP, TIP48, TIP49, hGCN5; upper panel) or acetylated forms of histone H3 (Acetyl-K9, Acetyl-K14; lower panel);

FIG. 5C illustrates the results of chromatin-immunoprecipitation assays performed on HTLV-1-infected HuT-102 lymphocytes using antibodies that recognize various Myc-interacting factors (TIP60, TRRAP, TIP48, TIP49, hGCN5; upper panel) or acetylated forms of histone H3 (Acetyl-K9, Acetyl-K14; lower panel);

FIG. 5D shows a diagram of GST-HTLV-1 p30^{II} fusion proteins and relative input levels of GST-HTLV-1 p30^{II} and GST-p30^{II} truncation mutants, Myc, and TIP60 proteins;

FIG. 5E shows the input for GST-pull-down experiments using HeLa extracts and purified recombinant GST-HTLV-1 p30^{II} or GST-p30^{II} (1-98), GST-p30^{II} (99-154), and GST-p30^{II} (155-241) truncated mutant proteins;

FIG. 5F shows the results from GST-pull-down experiments using HeLa extracts and purified recombinant GST-HTLV-1 p30^{II} or GST-p30^{II} (1-98), GST-p30^{II} (99-154), and GST-p30^{II} (155-241) truncated mutant proteins;

FIG. 5G illustrates the results of ChIP analyses of HTLV-1 p30^{II}-Myc/TIP60 transcription complexes recruited to Myc-responsive E-box elements within the genomic cyclin D2 promoter in cultured HTLV-1-infected ATLL patient (ATL-1) lymphocytes in which PCR analyses of ChIP products were carried-out using PRM and UTR oligonucleotide primer pairs;

FIG. 6A illustrates expression of HTLV-1 p30^{II}-GFP in transfected 293A fibroblasts visualized by fluorescence-microscopy;

FIG. 6B shows the results of ChIP analyses performed on 293A fibroblasts transfected with HTLV-1 p30^{II}-GFP using various antibodies against specific Myc-interacting proteins;

FIG. 6C illustrates expression of GFP in 293A fibroblasts transfected with a pcDNA3.1-GFP control and visualized by fluorescence-microscopy;

FIG. 6D shows the results of ChIP analyses performed on 293A fibroblasts transfected with a pcDNA3.1-GFP control using various antibodies against specific Myc-interacting proteins;

FIG. 6E shows the results of luciferase assays in which 293A Fibroblasts were co-transfected with a human cyclin D2 promoter-luciferase reporter construct, CMV-HTLV-1 HTLV-1 p30^{II}-GFP, and increasing amounts of CMV-TIP60 (wild-type) or CMV-TIP60_{ΔHAT} (error bars represent of standard deviations from duplicate experiments);

FIG. 6F shows the results of luciferase assays in which 293A Fibroblasts were co-transfected with a tk-promoter-renilla-luciferase reporter construct, CMV-HTLV-1 HTLV-1 p30^{II}-GFP, and increasing amounts of CMV-TIP60 (wild-type) or CMV-TIP60_{ΔHAT} (error bars represent of standard deviations from duplicate experiments);

FIG. 7A shows a graphical illustration of microarray gene expression analyses performed on 293A fibroblasts transfected with a CβS empty vector control or CMV-HTLV-1 p30^{II} (HA) using Affymetrix Human U133Plus 2.0 full-genomic chips in which transcriptional activation of cellular genes by HTLV-1 p30^{II} is expressed as Fold Activation relative to the empty CβS vector control;

FIG. 7B shows a graphical illustration of microarray gene expression analyses performed on 293A fibroblasts transfected with a CβS empty vector control, CMV-HTLV-1 p30^{II} (HA), or CMV-HTLV-1 p30^{II} (HA) +CMV-TIP60_{ΔHAT} using Affymetrix Human U133Plus 2.0 full-genomic chips in which transcriptional activation of cellular genes by HTLV-1 p30^{II} is expressed as Fold Activation relative to the empty CβS vector control and TIP60-dependent genes are identified based upon their transcriptional repression in the presence of the trans-dominant-negative TIP60_{ΔHAT} mutant;

FIG. 7C shows a graphical illustration of microarray gene expression analyses performed on 293A fibroblasts transfected with a CβS empty vector control or CMV-HTLV-1 p30^{II} (HA) using Affymetrix Human U133Plus 2.0 full-genomic chips in which transcriptional repression of cellular genes by HTLV-1 p30^{II} is expressed as Fold Repression relative to the empty CβS vector control;

FIG. 7D shows a graphical illustration of microarray gene expression analyses performed on 293A fibroblasts transfected with a CβS empty vector control, CMV-HTLV-1 p30^{II} (HA), or CMV-HTLV-1 p30^{II} (HA) +CMV-TIP60_{ΔHAT} using Affymetrix Human U133Plus 2.0 full-genomic chips in which transcriptional repression of cellular genes by HTLV-1 p30^{II} is expressed as Fold Repression relative to the empty CβS vector control and TIP60-dependent or TIP60-independent genes are identified based upon their transcriptional repression in the presence of the trans-dominant-negative TIP60_{ΔHAT} mutant;

FIG. 8A illustrates representative results from triplicate foci-formation assays using immortalized human WR^{-/-} fibroblasts transfected with CβS empty vector (3.0 μg), CMV-HTLV-1 p30^{II} (HA) (3.0 μg), CβF-FLAG-Myc (3.0 μg), and combinations of CβS (1.5 μg)/CβF-FLAG-Myc (3.0 μg) or CMV-HTLV-1 p30^{II} (HA) (1.5 μg)/CβF-FLAG-Myc (3.0 μg);

FIG. 8B is a bar graph of the results shown in FIG. 8A;

FIG. 8C shows micrographs of CβS/CβF-FLAG-Myc-transfected (upper panels) or CMV-HTLV-1 p30^{II} (HA)/CβF-FLAG-Myc-transfected (lower panels) immortalized human WRN^{-/-} fibroblasts viewed with phase contrast optics (left panels) or after staining with DAPI (middle panels) or a monoclonal anti-HA tag antibody and a rhodamine red-conjugated anti-mouse secondary antibody (right panels)

FIG. 8D shows HTLV-1 p30^{II} (HA)/Myc-transformed fibroblasts stained with DAPI (left panel) and a monoclonal anti-HA tag antibody and a rhodamine red-conjugated anti-mouse secondary antibody viewed in the blue channel (left panel), red channel (center panel), and both the blue and red channels (right panel);

FIG. 8E shows an increased number of multi-nucleated giant cells observed in isolated HTLV-1 p30^{II} (HA)/Myc-transformed WRN^{-/-} fibroblasts expanded in culture (arrows, micrograph) and expression of HTLV-1 p30^{II} (HA) detected by immunoblotting using a monoclonal anti-HA antibody (lower panel);

FIG. 9A shows the results of a first foci-formation/transformation assay using immortalized human WRN^{-/-} fibroblasts transfected with CβF-FLAG-Myc (3.0 μg) and either CMV-HTLV-1 p30^{II} (HA) or empty CβS vector control (1.5 μg) in the presence of CMV-TIP60, CMV-TIP60_{ΔHAT}, or CMV-TIP60_{L497A} (3.0 μg)

FIG. 9B shows the results of a second foci-formation/transformation assay using immortalized human WRN^{-/-} fibroblasts transfected with CβF-FLAG-Myc (3.0 μg) and either CMV-HTLV-1 p30^{II} (HA) or empty CβS vector control (1.5 μg) in the presence of CMV-TIP60, CMV-TIP60_{ΔHAT}, or CMV-TIP60_{L497A} (3.0 μg);

FIG. 9C shows that over-expression of wild-type TIP60 results in increased foci-formation in WRN^{-/-} fibroblasts co-transfected with CMV-HTLV-1 p30^{II} (HA), CβF-FLAG-Myc, and CMV-TIP60;

FIG. 9D shows that co-expression of the trans-dominant-negative TIP60_{ΔHAT} mutant in the cells of FIG. 9C inhibits cellular transformation by HTLV-1 p30^{II} (HA)/Myc;

FIG. 9E shows representative results from duplicate foci-formation/transformation assays using immortalized human WRN^{-/-} fibroblasts transfected as in FIGS. 9A and 9B in the presence of increasing amounts of CβS-TRRAPₐₙₜᵢ₋ₛₑₙₛₑ or CβS empty vector (0.5, 1.5, 3.0 μg) with an asterisk denoting HTLV-1 p30^{II} (HA)/Myc foci-formation; and

FIG. 10 illustrates a model of HTLV-1 p30^{II} modulatory interactions with Myc-TIP60 transcription complexes assembled on E-box enhancer elements within promoters of Myc-responsive genes (nucleosomal acetylation associated with transcriptional activation indicated).

### DETAILED DESCRIPTION OF THE INVENTION

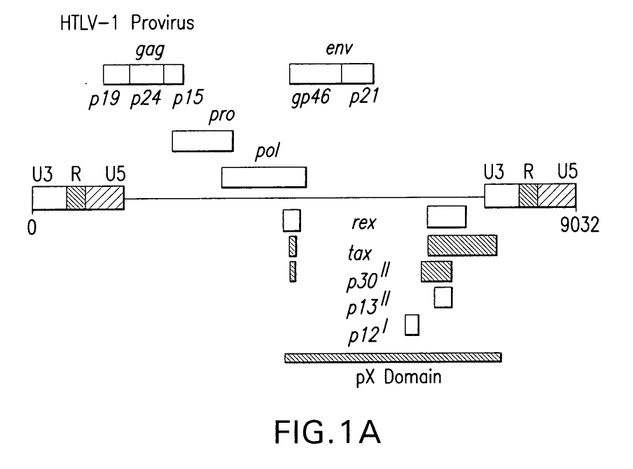

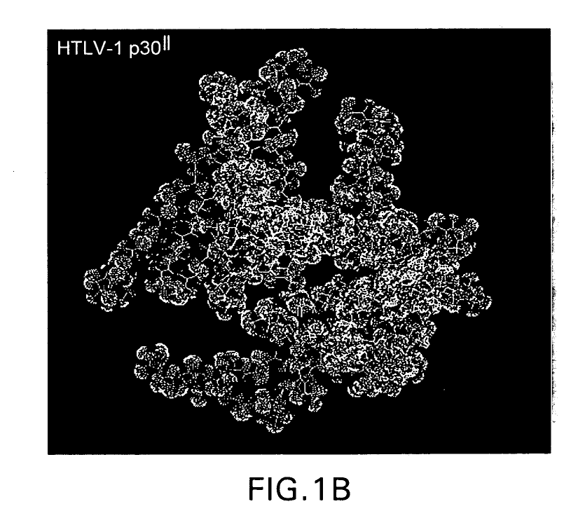

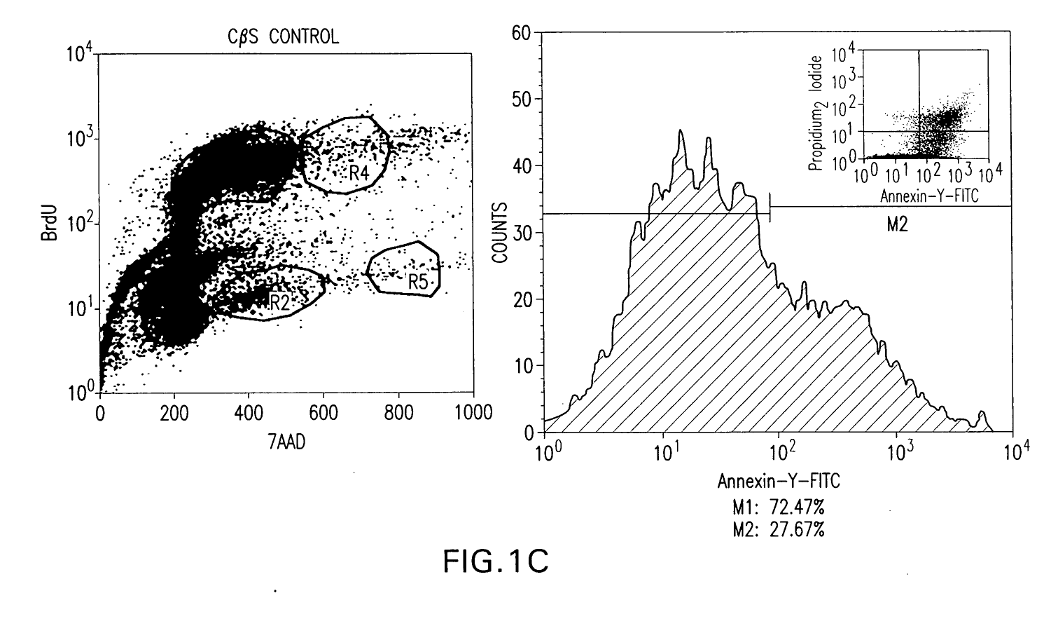

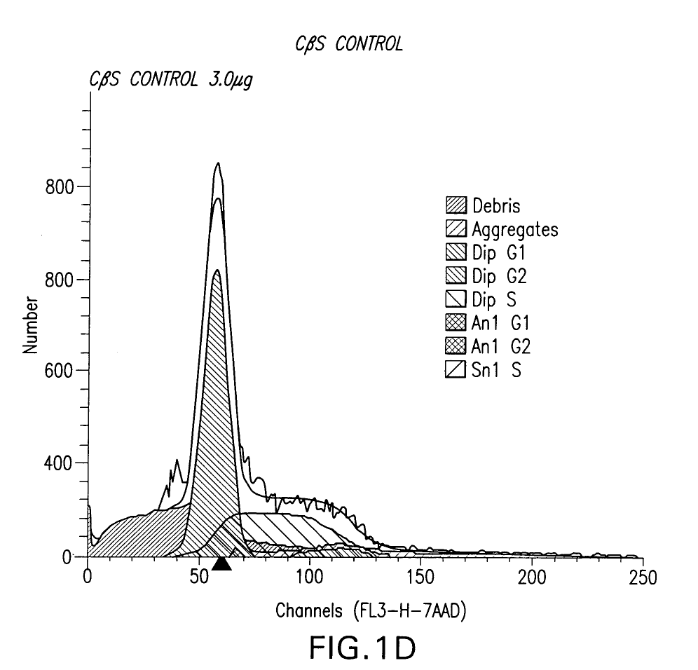

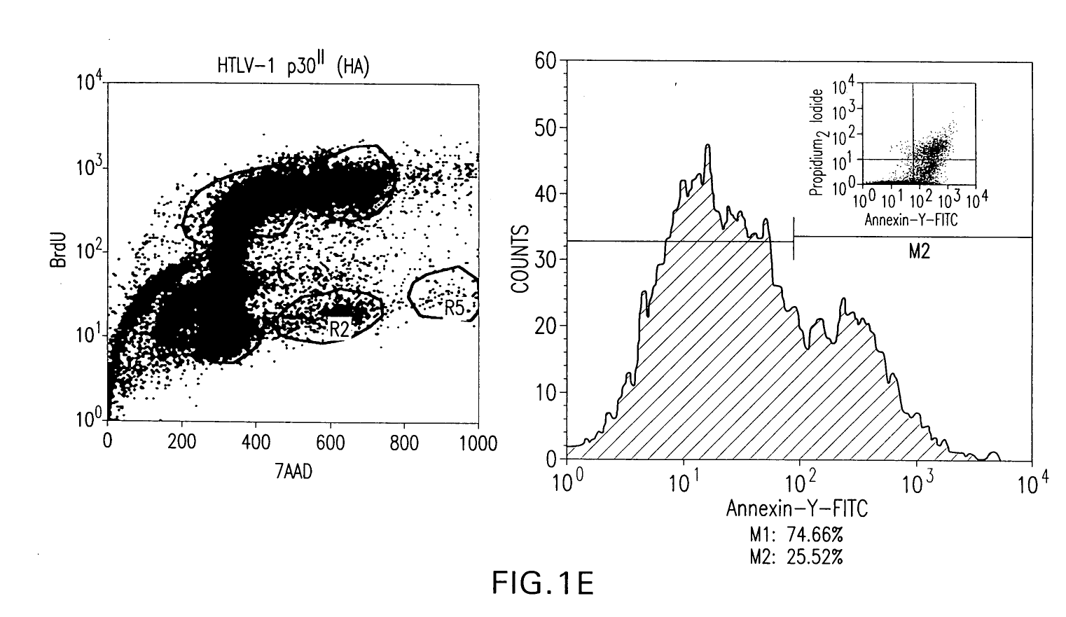

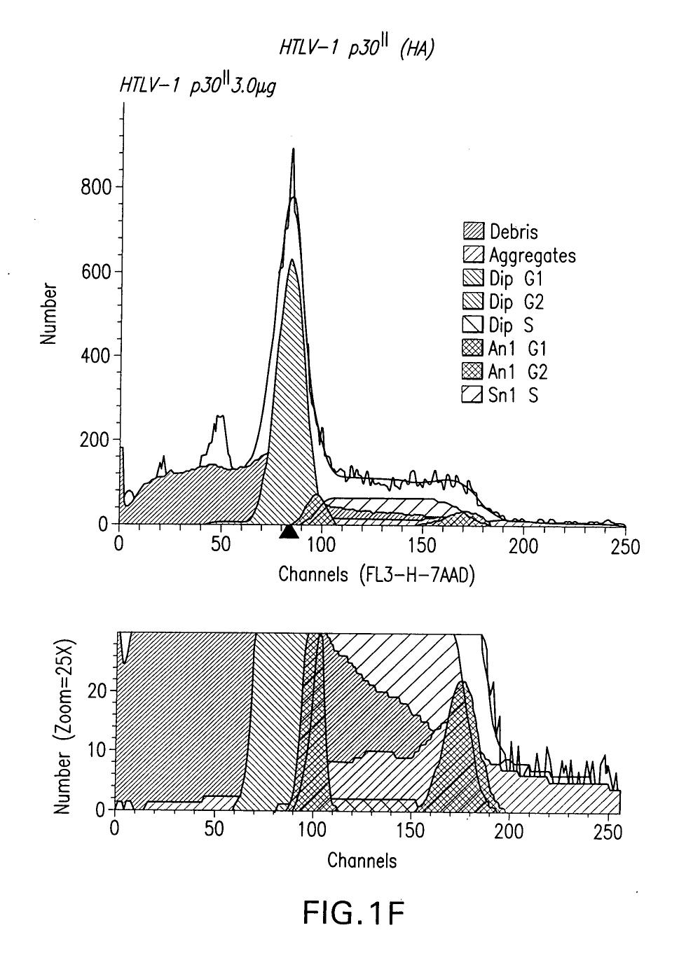

HTLV-1 p30^{II} increases S-phase progression and promotes polyploidy. The conserved pX domain of HTLV-1 encodes at least five non-structural regulatory factors, including the viral trans-activator, Tax, and an alternative splice-variant, p30^{II} (FIG. 1A). The HTLV-1 p30^{II} protein is comprised of 241 amino acid residues and contains Arg- and Ser/Thr-rich domains. RasMol structural prediction analyses (Brookhaven protein databank) indicate that p30^{II} possesses four alpha-helices and nineteen beta-sheet regions (FIG. 1B). The alpha-helices likely serve as interacting or docking sites for cellular factors, whereas the Ser/Thr-rich domains may provide targets for phosphorylation by kinases that modulate p30^{II}'s functions or interactions. As relatively little is known with respect to the functions of HTLV-1 pX accessory factors, such as p30^{II}, the issue of whether the p30^{II} protein contributes to lymphoproliferation in HTLV-1-infected T-cells by altering cell-cycle regulation was investigated.

To determine whether HTLV-1 p30^{II} influences cell-cycle progression and/or apoptosis, Molt-4 and Jurkat E6.1 lymphocytes were transfected with a CMV-HTLV-1 p30^{II} (HA) expression construct or an empty CβS vector control. Transfected cultures were assayed for bromodeoxyuridine (BrdU)-incorporation/cell-cycle progression or programmed cell-death using flow-cytometric analyses (FIGS. 1C to 1F and Tables 1-2). HTLV-1 p30^{II}-expressing cells exhibited markedly increased S-phase progression and significant polyploidy, as determined by BrdU-incorporation and 7-AAD-staining of total genomic DNA (FIGS. 1C and 1E, left panels, FIGS. 1D and 1F and Tables 1-2). However, p30^{II} did not induce apoptosis in transfected cells as determined by annexin-V-FITC/propidium iodide-staining and FACS (FIGS. 1C and 1E, right panels). These results suggest that p30^{II} may contribute to lymphoproliferation and genomic instability in HTLV-1-infected cells during ATLL by affecting S-phase regulatory factors, such as Myc and/or E2F.

TABLE 1

| | |
|---|---|
| Diploid: | 99.68% |
| Dip G1: | 60.50% at 58.18 |
| Dip G2: | 2.85% at 116.37 |
| Dip S: | 36.65% G2/G1: 2.00 |
| % CV: | 9.72 |
| Aneuploid 1: | 0.32% |
| An1 G1: | 66.73% at 67.97 |
| An1 G2: | 0.00% at 147.34 |
| An1 S: | 32.27% G2/G1: 2.17 |
| % CV: | 1.32 DI: 1.17 |
| Total Aneuploid S-Phase: | 33.27% |
| Total S-Phase: | 36.64% |
| Total B.A.D.: | 23.45% |
| Debris: | 29.64% |
| Aggregates: | 7.68% |
| Modeled events: | 23210 |
| All cycle events: | 14547 |
| Cycle events per channel: | 161 |
| RCS: | 3.365 |

TABLE 2

| | |
|---|---|
| Diploid: | 65.58% |
| Dip G1: | 98.05% at 85.10 |
| Dip G2: | 0.17% at 170.20 |
| Dip S: | 1.79% G2/G1: 2.00 |
| % CV: | 8.05 |
| Aneuploid 1: | 34.42% |
| An1 G1: | 11.34% at 99.15 |
| An1 G2: | 11.09% at 174.33 |
| An1 S: | 77.57% G2/G1: 1.76 |
| % CV: | 4.05 DI: 1.17 |
| Total Aneuploid S-Phase: | 77.57% |
| Total S-Phase: | 27.87% |
| Total B.A.D.: | 28.92% |
| Debris: | 41.67% |
| Aggregates: | 6.66% |
| Modeled events: | 22920 |
| All cycle events: | 11843 |
| Cycle events per channel: | 131 |
| RCS: | 3.211 |

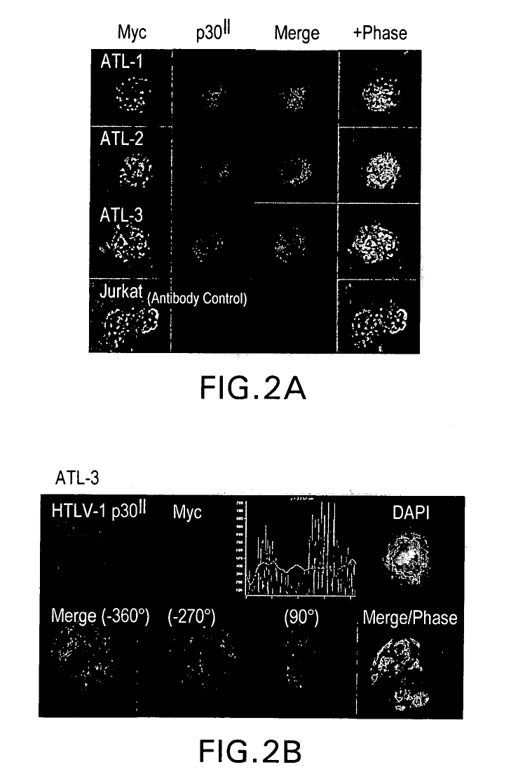

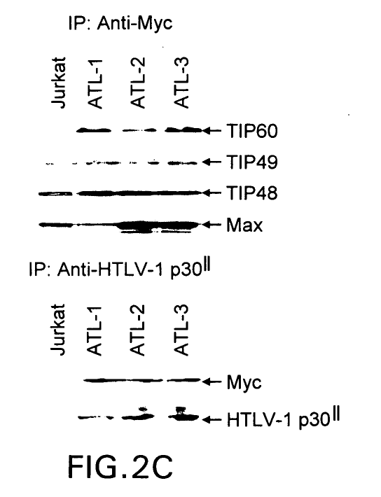

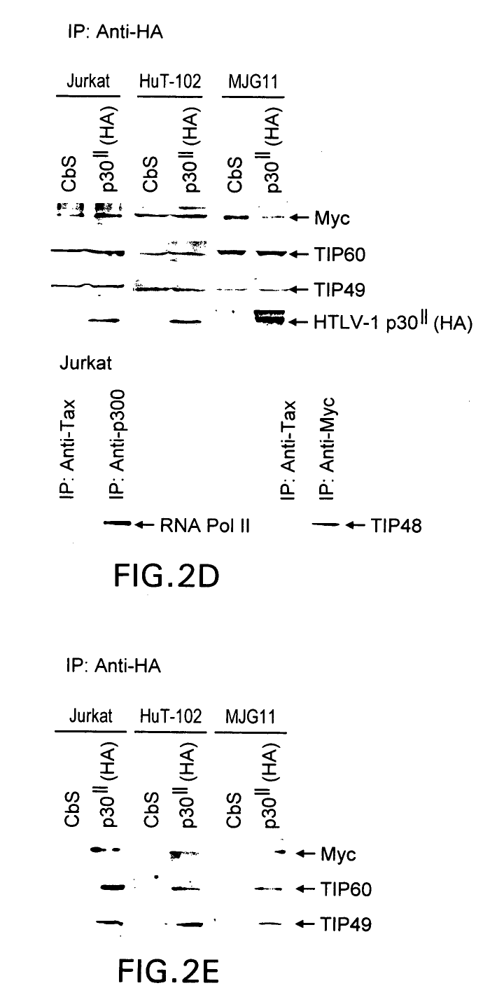

The HTLV-1 p30^{II} protein interacts in Myc-TIP60 immune-complexes in ATLL patient lymphocytes. The p30^{II} protein was detected in cultured HTLV-1-infected lymphocytes, derived from three different ATLL patients (ATL-1, ATL-2, ATL-3) diagnosed with clinical acute-stage leukemias, by immunofluorescence-laser confocal microscopy (FIGS. 2A and 2B) and immuno-blotting (FIG. 2C). Three-dimensional Z-stack composite images of ATL-3 demonstrate that p30^{II}/Myc proteins co-localize in the nucleus in all focal planes in HTLV-1-infected cells (FIG. 2A). Relative fluorescence-intensities for p30^{II}/Myc-specific signals and DAPI nuclear-staining are shown for reference (FIG. 2B). HTLV-1 p30^{II} is present in Myc-containing immunoprecipitated complexes in ATLL patient lymphocytes (FIG. 2C). Intriguingly, immunoprecipitation of Myc revealed that TIP49 (RUVBL1), TIP48 (RUVBL2), and Max are present bound to Myc, but the TIP60 histone acetyltransferase (HAT) was not detected in Myc-containing co-immune complexes in uninfected Jurkat E6.1 lymphocytes (FIG. 2C). The NH₂-terminus of Myc is essential for Myc-dependent transformation and apoptosis-inducing functions and contains two conserved Myc homology domains (Myc boxes I and II, MBI and MBII, respectively) that interact with cellular factors. The transcriptional coactivator, TRRAP/p434, and the ATPases/helicases, TIP 49 (RUVBL1) and TIP 48 (RUVBL2), interact with amino acids within MBII. To determine if HTLV-1 p30^{II} interacts with known Myc-binding partners, Jurkat E6.1 lymphocytes or HTLV-1-infected Hut-102 and MJ[G11] lymphocytes were transfected with CMV-HTLV-1 p30^{II} (HA) or an empty CβS vector control and co-immunoprecipitations using a monoclonal anti-HA antibody were performed (CA5, Roche Molecular Diagnostics). As shown in FIGS. 2D and 2E, HTLV-1 p30^{II} (HA) immunoprecipitates with Myc, TRRAP, TIP60, and TIP49 (RUVBL1). However, TIP48 (RUVBL2) and RNA Pol II were not detected in anti-HA immunoprecipitates, although both proteins were detected in control immunoprecipitations using antibodies against known interacting proteins (FIG. 2D, lower panels). These data suggest that HTLV-1 p30^{II} may modulate Myc functions through interactions with Myc-associated transcriptional coactivators on promoters of responsive genes.

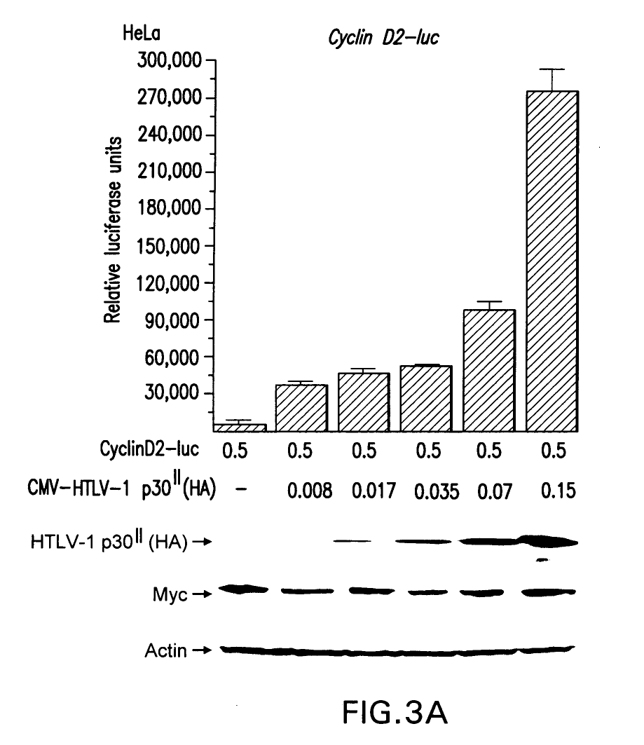

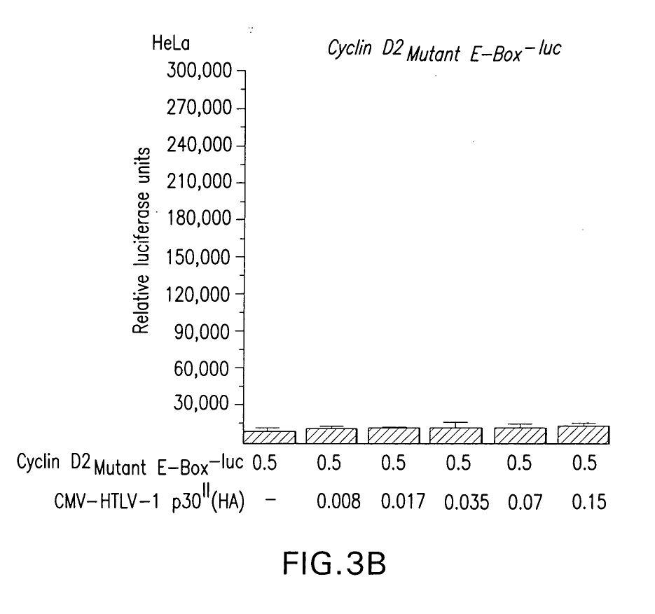

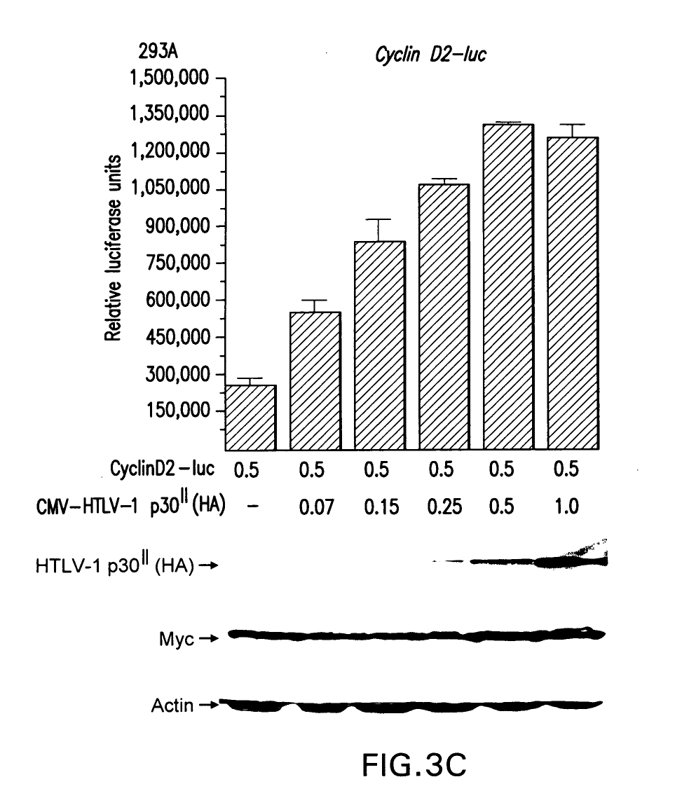

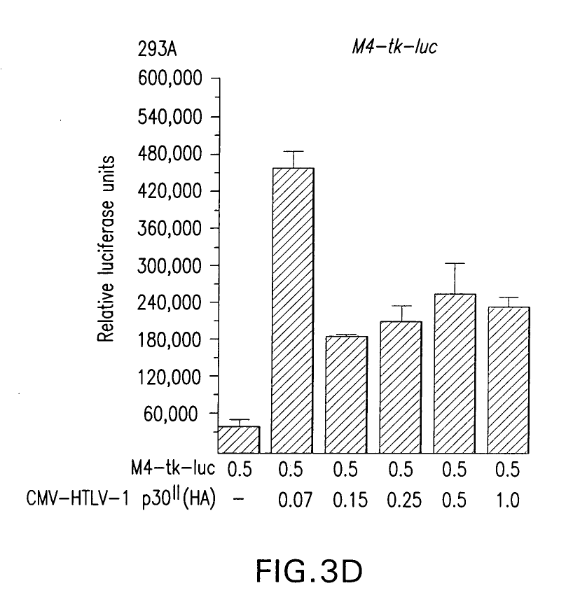

HTLV-1 p30^{II} trans-activates Myc-responsive E-box elements within the human cyclin D2 promoter. To investigate the possibility that HTLV-1 p30^{II} might affect Myc-dependent transcription, HeLa cells were co-transfected with a human cyclin D2 promoter-luciferase reporter construct, containing two conserved Myc-responsive E-box enhancer elements (CACGTG), in the presence of increasing amounts of CMV-HTLV-1 p30^{II} (HA). Results shown in FIG. 3A demonstrate that HTLV-1 p30^{II} significantly trans-activates the human cyclin D2 promoter in a dose-dependent manner. A mutant cyclin D2 promoter, lacking Myc-responsive E-box elements, was not transcriptionally activated by p30^{II} indicating that p30^{II}-mediated trans-activation from the human cyclin D2 promoter requires the conserved Myc-responsive E-box enhancer elements (FIGS. 3A and 3B). The HTLV-1 p30^{II} (HA)-tagged protein was detected in transfected cells by immunoblotting using a monoclonal anti-HA antibody (CA5. Roche Molecular Biochemicals) (FIG. 3A). Intracellular levels of Myc were not altered by HTLV-1 p30^{II} expression (FIG. 3A, lower panels). HTLV-1 p30^{II} also transcriptionally activates the human cyclin D2 promoter in transfected 293A fibroblasts in a dose-dependent manner (FIG. 3C). To confirm that HTLV-1 p30^{II} promotes Myc-dependent transcription from E-box enhancer elements, 293A fibroblasts and HeLa cells were co-transfected with a synthetic tk minimal promoter-luciferase reporter construct (M4-tk-luc) that contains four tandem E-boxes. As shown in FIG. 3D, HTLV-1 p30^{II} trans-activates E-box enhancer elements within M4-tk-luc suggesting that p30^{II} promotes S-phase progression through Myc-dependent transcriptional interactions.

Interestingly, p30^{II}, at the lowest concentration used, induced approx 13-fold trans-activation from the synthetic M4-tk-luc promoter, whereas higher concentrations induced lower (5 to 7-fold) levels of transcriptional activation (FIG. 3D). These observations are consistent with findings by Zhang et al. (2000, J. Virol. 74:11270-11277) demonstrating that p30^{II}-dependent trans-activation from the HTLV-1 promoter (Tax-responsive elements, TREs) maximally occurs at low p30^{II} concentrations and diminishes with increased p30^{II} expression.

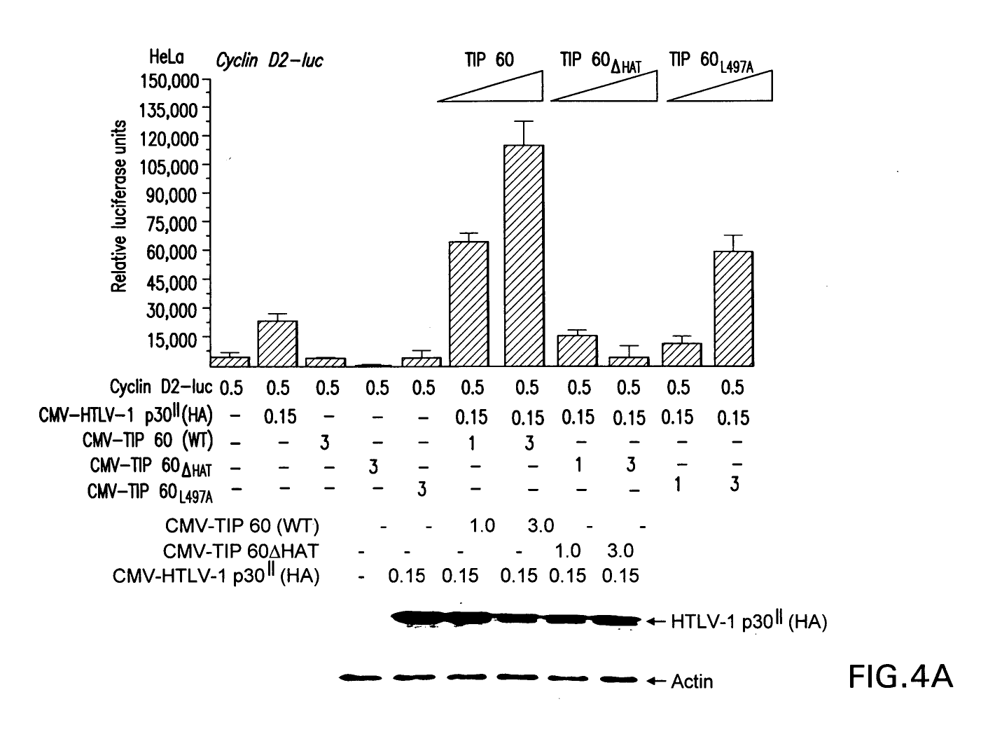

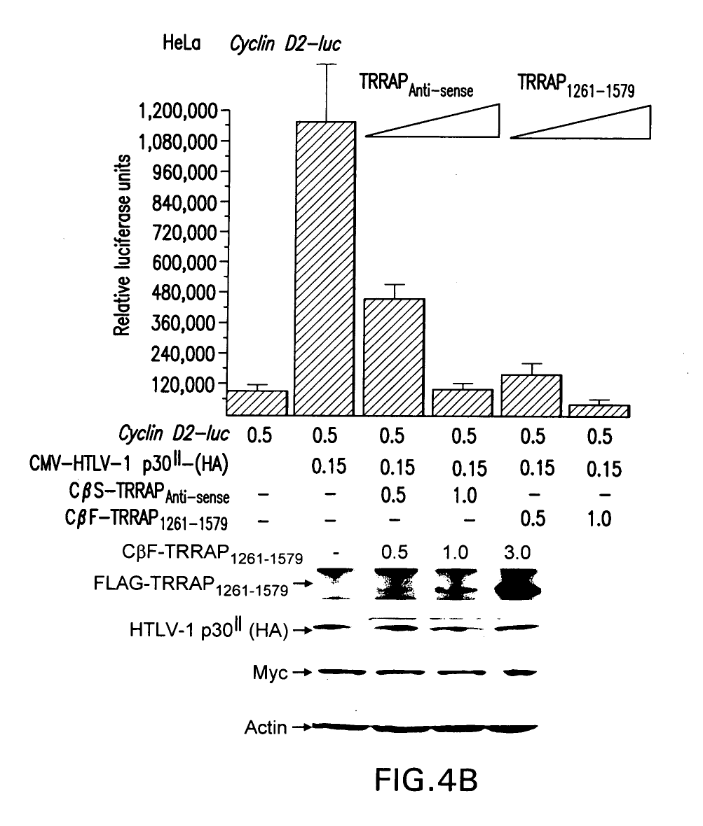

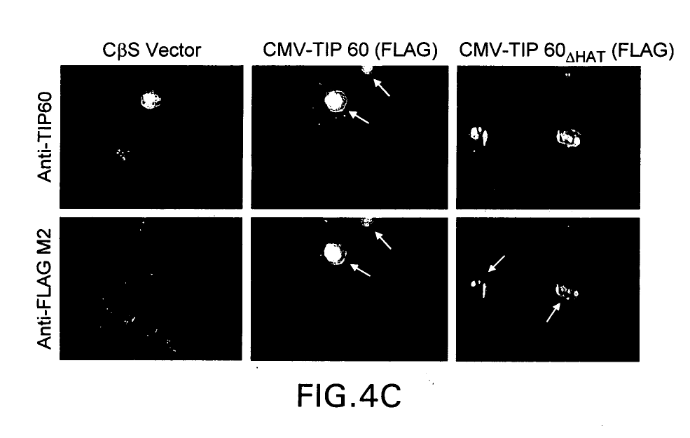

Transcriptional activation by HTLV-1 p30^{II} is dependent upon the TIP60 and TRRAP/p434 coactivators. Since Myc may interact with the transcriptional coactivator/HAT, TIP60, c-Myc may be a substrate for lysine-acetylation by TIP60, and Myc may interact in chromatin-remodeling complexes with the ATM-related TRRAP/p434 protein, tests were performed to determine whether HTLV-1 p30^{II}-mediated trans-activation requires TIP60 and TRRAP/p434 functions. HeLa cells were co-transfected with a human cyclin D2 promoter-luciferase reporter construct and CMV-HTLV-1 p30^{II} (HA) in the presence of increasing amounts of CMV-TIP60, CMV-TIP60_{ΔHAT} (a trans-dominant negative HAT-inactive mutant), or CMV-TIP⁶⁰_{L497A}—a carboxyl-terminal mutant impaired for interactions with cellular factors, including the androgen receptor. Ectopic expression of TIP60 alone did not significantly trans-activate the human cyclin D2 promoter, however, TIP60 over-expression enhanced HTLV-1 p30^{II}-mediated trans-activation in a dose-dependent manner (FIG. 4A). The trans-dominant negative TIP60_{ΔHAT} mutant potently inhibited p30^{II}-mediated transcriptional activation (FIG. 4A), suggesting that HTLV-1 p30^{II} trans-activation requires TIP60-associated HAT activity. The TIP60_{L497A} mutant also weakly enhanced p30^{II}-mediated trans-activation (FIG. 4A). Over-expression of wild-type TIP60 or the trans-dominant-negative TIP⁶⁰_{ΔHAT} mutant did not alter expression of the HTLV-1 p30^{II} (HA) protein in transfected HeLa cells (FIG. 4A, lower panels). Inhibition of TRRAP/p434, as a result of co-expressing either TRRAPₐₙₜᵢ₋ₛₑₙₛₑ RNA or a trans-dominant negative TRRAP mutant, TRRAP₁₂₆₁₋₁₅₇₉ (FLAG-epitope-tagged), prevented HTLV-1 p30^{II}-mediated transcriptional activation from the human cyclin D2 promoter (FIG. 4B). The trans-dominant-negative, FLAG-tagged TRRAP₁₂₆₁₋₁₅₇₉ protein did not alter the expression of HTLV-1 p30^{II} (HA) (FIG. 4B, lower panels). Immunofluorescence-microscopy was then performed, using a monoclonal anti-FLAG M2 antibody (Sigma Chemical Corp.) and a rabbit polyclonal anti-TIP60 antibody (Upstate Biotechnology), to visualize expression of the FLAG-tagged wild-type TIP60 or TIP60_{ΔHAT} proteins relative to endogenous TIP60. Results in shown FIG. 4C demonstrate that the FLAG-tagged TIP60 proteins were drastically over-expressed relative to endogenous TIP60 in transfected cells. These data collectively indicate that HTLV-1 p30^{II} synergizes with the TIP60 HAT to trans-activate Myc-responsive E-box elements within the human cyclin D2 promoter, requiring the transcriptional coactivator TRRAP/p434.

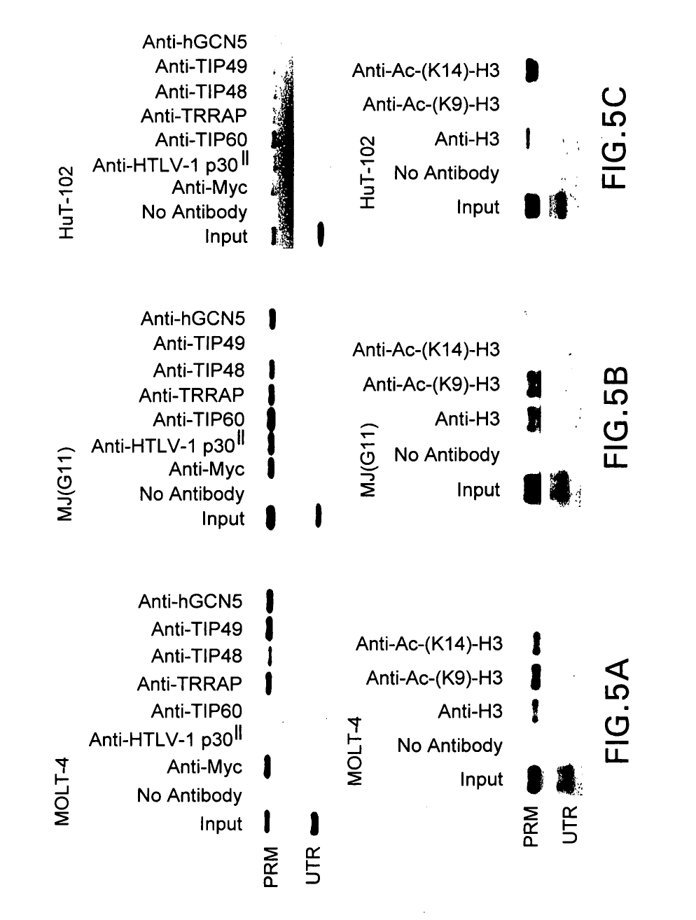

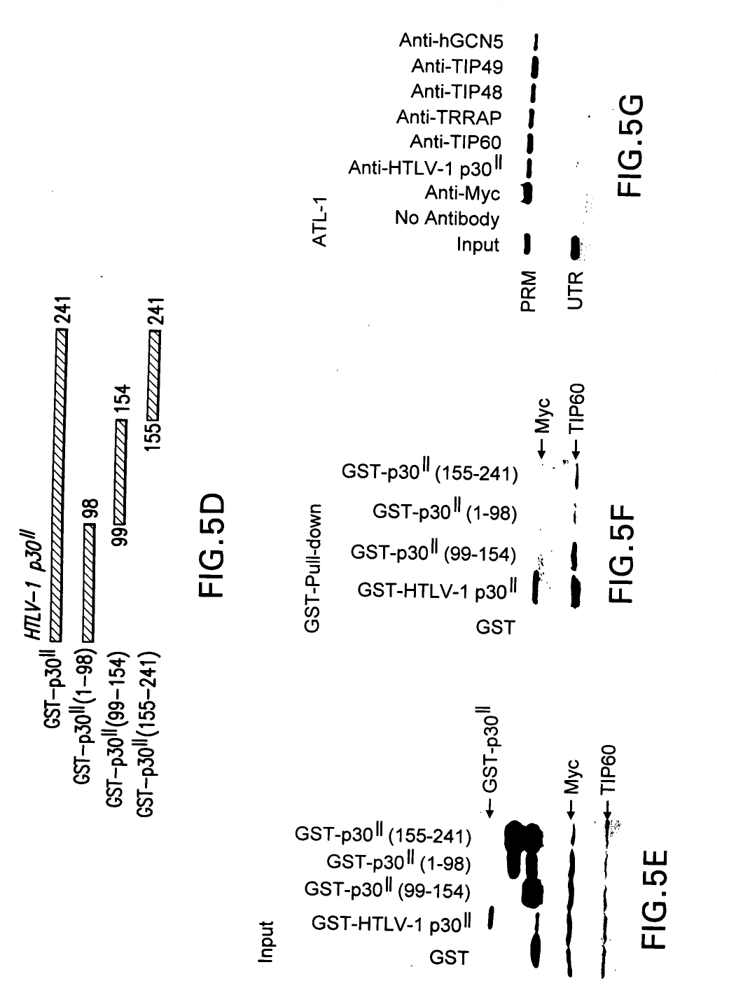

HTLV-1 p30^{II} stabilizes Myc/TIP60 chromatin-remodeling transcription complexes in HTLV-1-infected lymphocytes. Since HTLV-1 p30^{II} transcriptionally activates the conserved Myc-responsive E-box enhancer elements within the human cyclin D2 promoter (FIGS. 3A and 3C), a chromatin-immunoprecipitation (ChIP) procedure (described in Vervoorts et al., 2003, EMBO Rep. 4:484-490) was used to determine whether p30^{II} is present in Myc-containing chromatin-remodeling complexes. Formaldehyde-cross-linked genomic DNA complexes in uninfected Molt-4 lymphocytes or HTLV-1-infected MJ[G11] and HuT-102 lymphocytes were fragmented by sonication and oligonucleosomal-protein complexes were precipitated using antibodies against candidate Myc-binding factors. Cross-links were reversed and specific oligonucleotide DNA primer pairs were used in PCR reactions to amplify immunoprecipitated DNA regions spanning conserved E-box elements (PRM) or an untranslated sequence (UTR) as negative control. Results shown in FIGS. 5A to 5C (top panels) demonstrate that HTLV-1 p30^{II} was only detected bound to E-box enhancer elements in HTLV-1-infected lymphocytes. Myc, TRRAP, TIP49 (RUVBL1), TIP48 (RUVBL2), and the acetyltransferase hGCN5 were present in chromatin-remodeling complexes in uninfected Molt-4 cells and in HTLV-1-infected MJ[G11] and HuT-102 lymphocytes (FIGS. 5A to 5C, top panels). Surprisingly, TIP60 was only detected in Myc-containing transcription complexes that contained p30^{II} in HTLV-1-infected T-cells (FIGS. 5A to 5C, top panels), consistent with co-immunoprecipitation results and observed effects of ectopic TIP60 in trans-activation assays (see FIGS. 2B, 4A). The diminished recruitment of TIP49 to Myc-containing transcription complexes on the cyclin D2 promoter in HTLV-1-infected MJ[G11] cells was not attributable to apparent differences in p30^{II}/Myc/TIP60 interactions (FIG. 5A). Histone H3-acetylation surrounding the E-box enhancer elements within the human cyclin D2 promoter, consistent with transcriptional activation, was detected in all cell-types, with the exception that H3 appeared to be differentially-acetylated on Lys-9 and Lys-14 residues in HTLV-1-infected MJ[G11] and HuT-102 cells, respectively (FIG. 5A, lower panels). Differences in histone H3-acetylation, however, did not correlate with the stabilization of p30^{II}/Myc/TIP60 transcriptional interactions in HTLV-1-infected T-cell-lines.

To identify residues within HTLV-1 p30^{II} that interact with Myc/TIP60 complexes in vivo, a panel of pGEX 4T.1-glutathione S-transferase (GST)-HTLV-1 p30^{II} constructs, expressing full-length GST-HTLV-1 p30^{II} or various truncation mutants, GST-p30^{II} (residues 1-98), GST-p30^{II} (residues 99-154), GST-p30^{II} (residues 155-241) spanning the entire coding region of HTLV-1 p30^{II} were generated (FIG. 5D, see diagram). These proteins were expressed in E. coli, BL21, bacteria and purified recombinant GST-HTLV-1 p30^{II} fusion proteins were used in GST-pull-down experiments as described in Harrod et al. (1998, Mol. Cell Biol. 18:5052-5061). GST-proteins were incubated with HeLa nuclear extracts at 4° C. overnight and complexes were precipitated with glutathione-Sepharose 4B (Amersham-Pharmacia Biotech). The matrices were washed and bound factors were eluted using 10 mM reduced glutathione buffer. Input levels of purified recombinant GST or GST-HTLV-1 p30^{II} proteins, Myc, and TIP60 are shown in FIGS. 5E and 5F. Results in FIG. 5F demonstrate that full-length GST-HTLV-1 p30^{II} interacts with both Myc and TIP60 in HeLa nuclear extracts. Deletion of amino acid residues from either the NH₂-terminus or COOH-terminus of p30^{II}, disrupts Myc-binding, however, the TIP60-interacting region of HTLV-1 p30^{II} was mapped to residues between positions 99-154 (FIG. 5B).

Recruitment of HTLV-1 p30^{II}/Myc/TIP60 chromatin-remodeling complexes to conserved, Myc-responsive E-box enhancer elements within the cyclin D2 promoter in cultured HTLV-1-infected ATLL patient lymphocytes (ATL-1) was examined next. Chromatin-immunoprecipitations were performed using antibodies that recognize endogenous HTLV-1 p30^{II}, Myc, and known Myc-interacting factors as described. Polymerase chain-reaction amplification of ChIP products was performed using the PRM and UTR oligonucleotide DNA primer pairs. Results shown in FIG. 5G demonstrate that p30^{II} is present in Myc/TIP60 transcription complexes assembled on E-box enhancer elements within the cyclin D2 promoter in HTLV-1 ATLL patient lymphocytes. The transcriptional coactivators, TRRAP/p434, TIP48, TIP49, and hGCN5 were also detected in p30^{II}/Myc/TIP60/cyclin D2 promoter complexes (FIG. 5G).

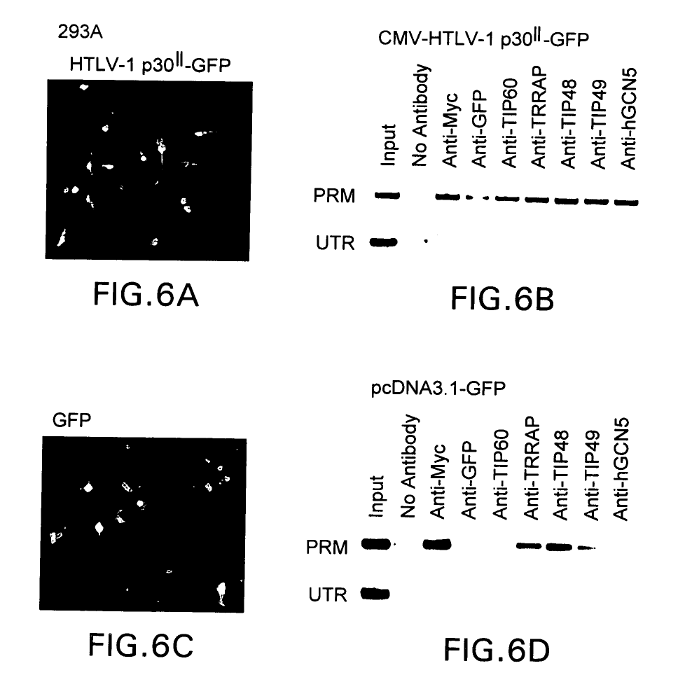

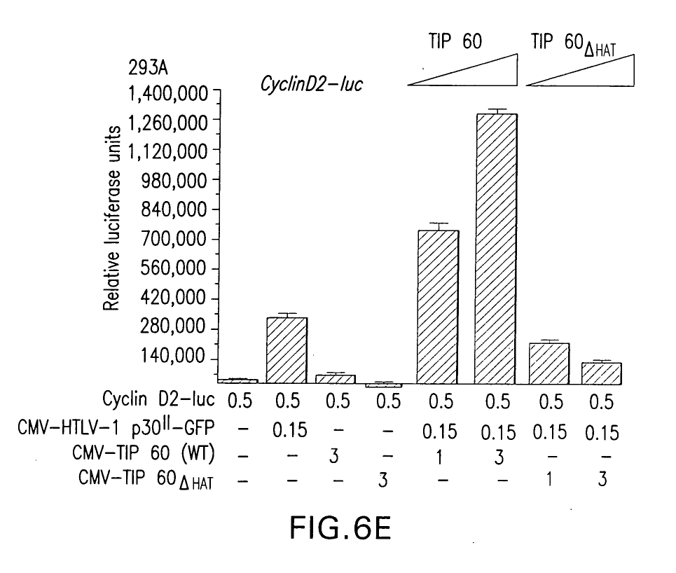

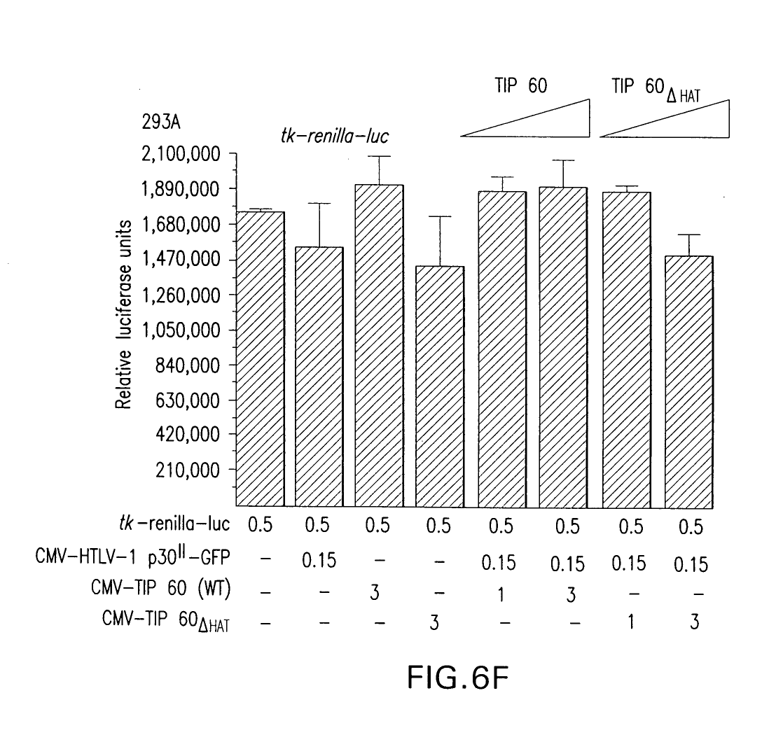

HTLV-1 p30^{II}-GFP stabilizes Myc/TIP60 interactions and trans-activates the cyclin D2 promoter in a TIP60 HAT-dependent manner. An HTLV-1 p30^{II}-green fluorescent protein (GFP) that is functionally identical to HTLV-1 p30^{II} (HA) was used to determine whether HTLV-1 p30^{II} similarly interacts in Myc/TIP60 transcription complexes in 293A fibroblasts. These cells were co-transfected 293A with CMV-HTLV-1 p30^{II}-GFP or a pcDNA3.1-GFP vector control and ChIP analyses were performed. Nucleoprotein complexes were cross-linked by treatment with formaldehyde and oligonucleosomal fragments were generated by brief sonication of extracted genomic DNA. Chromatin-immunoprecipitations were performed as described and ChIP products were amplified by PCR using the PRM and UTR oligonucleotide DNA primer pairs. Similar expression of HTLV-1 p30^{II}-GFP and GFP proteins was visualized in transfected 293A fibroblasts by fluorescence-microscopy (FIGS. 6A and 6C). The HTLV-1 p30^{II}-GFP protein was immunoprecipitated, bound to Myc-containing transcription complexes on conserved E-box elements within the cyclin D2 promoter in transfected 293A fibroblasts, using an anti-GFP antibody (FIG. 6B). No ChIP product was detected for the anti-GFP immunoprecipitation in 293A cells transfected with the pcDNA3.1-GFP control (FIG. 6D). While the transcriptional coactivators TRRAP/p434, TIP48, TIP49, and hGCN5 were present in Myc-containing complexes in both HTLV-1 p30^{II}-GFP and GFP-expressing cells, the TIP60 HAT was predominantly detected in HTLV-1 p30^{II}-GFP/Myc/TIP60 complexes (compare FIGS. 6B and 6D). However, TIP60 was weakly present in Myc-containing ChIP complexes in GFP-expressing cells consistent with the demonstration of pre-existing Myc-TIP60 interactions by Frank et al. (2003, EMBO Rep. 4:575-580) and Patel et al. (2004, Mol. Cell Biol. 24:10826-10834) (FIGS. 6C and 6D).

To determine whether the HTLV-1 p30^{II}-GFP protein also transcriptionally activates the human cyclin D2 promoter in a TIP60-dependent manner, 293A fibroblasts were co-transfected with a tk promoter-renilla-luciferase plasmid, a human dyclin D2 promoter-luciferase reporter plasmid and CMV-HTLV-1 p30^{II}-GFP in the presence of increasing amounts of either CMV-TIP60 (wild-type) or CMV-TIP60_{ΔHAT}, which expresses a trans-dominant-negative TIP60 mutant. Results shown in FIG. 6E demonstrate that HTLV-1 p30^{II}-GFP transcriptionally activates the human cyclin D2 promoter approximately 14-fold in transfected 293A fibroblasts compared to an empty pcDNA3.1-GFP control. Over-expression of wild-type TIP60, in the presence of HTLV-1 p30^{II}-GFP, significantly increased p30^{II}-GFP-dependent transcriptional activity in a dose-dependent manner (FIG. 6E). Co-expression of the trans-dominant-negative TIP60_{ΔHAT} mutant repressed p30^{II} -GFP-dependent trans-activation from the human cyclin D2 promoter (FIG. 6E), consistent with results in FIG. 4A and an essential role for the TIP60 HAT in HTLV-1 p30^{II} transcriptional activation. Relative renilla-luciferase activities for each sample are shown in FIG. 6F for comparison of similar transfection efficiencies.

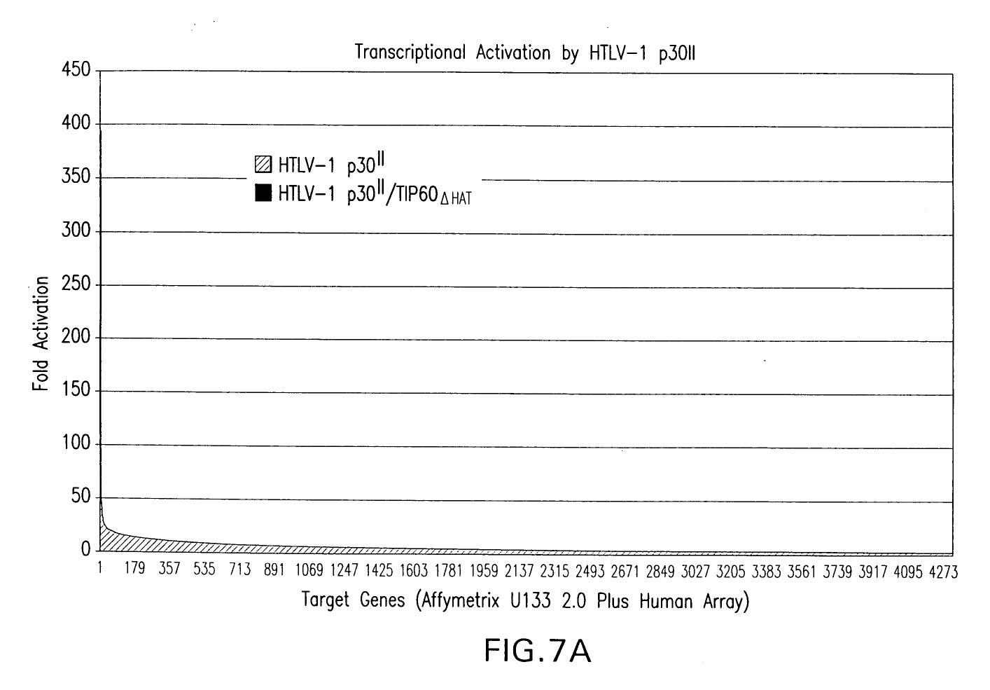

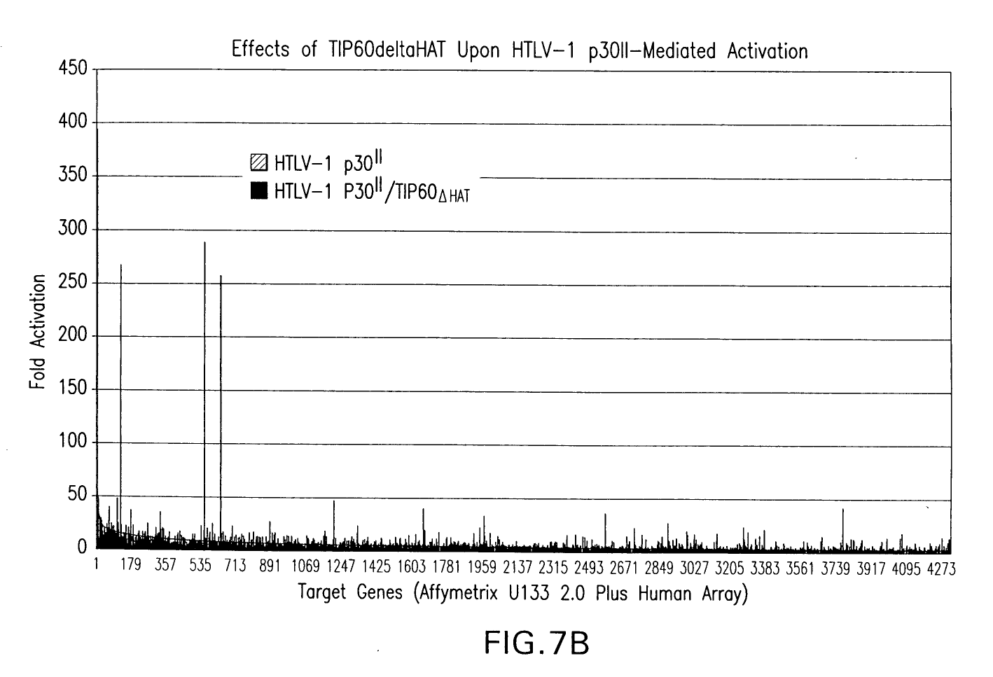

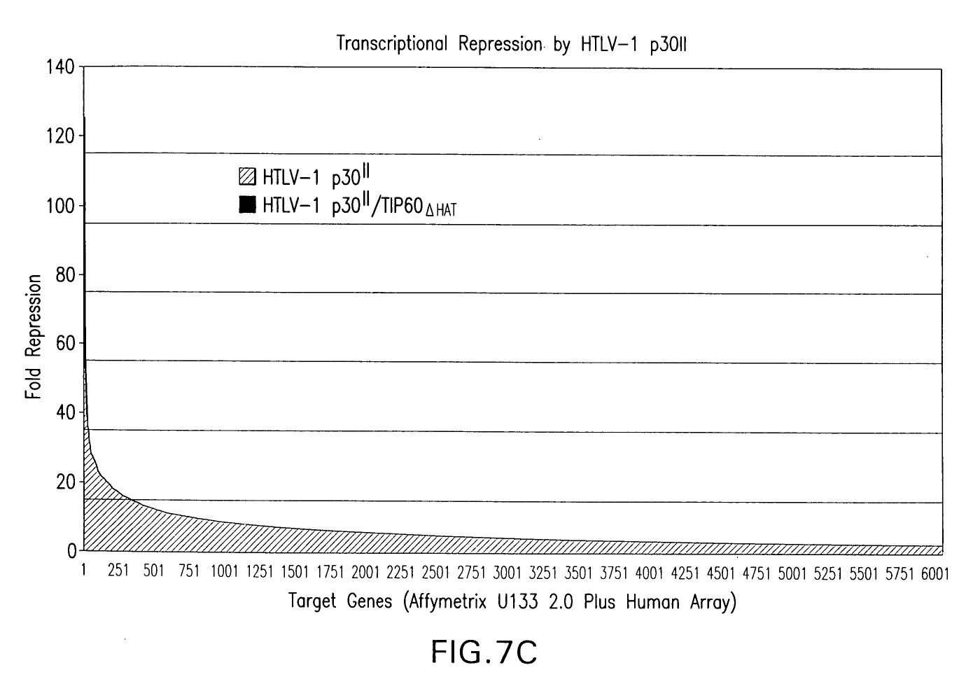

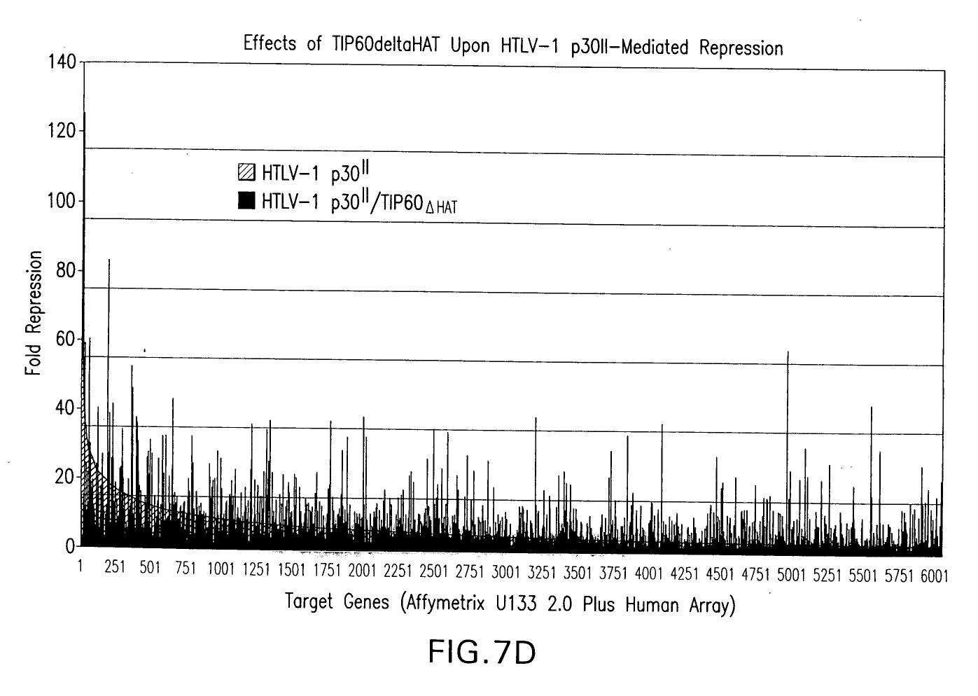

HTLV-1 p30^{II} transcriptionally activates numerous cellular genes in a TIP60-dependent or TIP60-independent manner. To comprehensively identify cellular gene sequences whose expression is altered by HTLV-1 p30^{II}-TIP60 transcriptional interactions, 293A fibroblasts were co-transfected with a COS empty vector control, CMV-HTLV-1 p30^{II} (HA), or CMV-HTLV-1 p30^{II} (HA) +TIP60_{ΔHAT} which expresses a trans-dominant-negative mutant that interferes with endogenous TIP60 functions. Total cellular RNAs were extracted and microarray gene expression analyses were performed using Affymetrix Human U133Plus 2.0 full-genomic chips. Transcriptional activation of cellular target genes is expressed as Fold-Activation relative to the empty CβS vector control and the lower-limit for trans-activation was set at 2.5-fold. FIG. 7A shows a graphical representation of cellular target genes transcriptionally activated by HTLV-1 p30^{II} (HA) (red lines). TIP60-dependent gene sequences were identified based upon their transcriptional repression in the presence of the TIP60_{ΔHAT} mutant and are indicated by green lines (FIG. 7A). In general, the fold trans-activation by HTLV-1 p30^{II} (HA) ranged between 2.5-fold to 393-fold for specific target genes (FIG. 7A). Numerous cellular genes are also transcriptionally repressed as a result of HTLV-1 p30^{II} expression. Results in FIG. 7B are a graphical representation of cellular target genes transcriptionally repressed (with levels ranging between 2.5-fold to 125-fold trans-repression) by HTLV-1 p30^{II} (HA) (red lines). Effects of the trans-dominant-negative TIP60 HT mutant upon transcriptional repression by HTLV-1 p30^{II} (HA) are indicated by green lines (FIG. 7B) Table 3 is a representative list of the major target gene sequences that are transcriptionally activated by HTLV-1 p30^{II} (HA) as determined by Affymetrix microarray gene expression analyses. TIP60-dependent gene sequences are indicated. Numerous cellular genes were transcriptionally induced by HTLV-1 p30^{II} (HA) in a TIP60-dependent or TIP60-independent manner, suggesting that p30^{II} may participate in multiple, distinct transcription complexes (Table 3).

TABLE 3
Target sequences transcriptionally-activated by
HTLV-1 p30″ (HA) in a TIP60-dependent or TIP60-independent
manner

| | HTLV-1 | | TIP60- |
|---|---|---|---|
| HTLV-1 | p30^{II}/ | | Depen- |
| p30^{II} | TIP60_{ΔHAT} | Gene or Sequence Identity | dent |
| 393.8725 | 396.7248 | TITLE = zinc finger protein | |
| | | 236 /DEF = Homo sapiens cDNA | |
| | | FLJ20840 fis, clone | |
| | | ADKA02336. | |
| 69.33333 | 2.666667 | Homo sapiens, clone | Yes |
| | | IMAGE: 4813412, mRNA | |
| 65.5 | 7 | Hs.42369 /UG_TITLE = ESTs | Yes |
| 56 | 46.4 | UG = Hs.66114 /UG_TITLE = ESTs | |
| 52.75 | 1 | CPX chromosome region, | Yes |
| | | candidate 1 /DEF = Homo | |
| | | sapiens cDNA FLJ25780 fis, | |
| | | clone TST06618. | |
| 49.09091 | 0.909091 | Hs.131856 /UG_TITLE = ESTs | Yes |
| 48 | 43 | Hs.23196 /UG_TITLE = ESTs | |
| 45.4 | 43.06667 | Hs.116301 /UG_TITLE = ESTs | |
| 40.44444 | 18.22222 | Hs.200286 /UG_TITLE = ESTs | Yes |
| 40.16667 | 22 | Homo sapiens, clone | Yes |
| | | IMAGE: 4812574, mRNA. | |
| 34.625 | 49.375 | Homo sapiens, clone | |
| | | IMAGE: 5172609, mRNA. | |
| 34.44444 | 3.444444 | Hs.122442 /UG_TITLE = ESTs | Yes |
| 31.85714 | 3.857143 | Homo sapiens cystic | Yes |
| | | fibrosis transmembrane | |
| | | conductance regulator | |
| | | isoform 36 (CFTR) mRNA, | |
| | | partial cds. | |
| 31.1875 | 1 | Homo sapiens myeloid cell | Yes |
| | | nuclear differentiation | |
| | | antigen (MNDA), mRNA | |
| 31 | 2.75 | Hs.145611 /UG_TITLE = ESTs | Yes |
| 30.88889 | 3.555556 | Hs.120414 /UG_TITLE = ESTs | Yes |
| 28.06667 | 10.66667 | Hs.125291 /UG_TITLE = ESTs | Yes |
| 27.72222 | 1.722222 | H. sapiens mRNA HTPCRX06 for | Yes |
| | | olfactory receptor. | |
| 27.625 | 28 | Homo sapiens, clone | |
| | | IMAGE: 5223057, mRNA. | |
| 26.65217 | 7.73913 | TESTI2017113. | Yes |
| 26.1875 | 1.5625 | protocadherin 15 /DEF = Homo | Yes |
| | | sapiens mRNA; cDNA | |
| | | DKFZp667A1711 (from clone | |
| | | DKFZp667A1711). | |
| 25.5 | 1.333333 | Hs.279616 /UG_TITLE = ESTs, | Yes |
| | | Highly similar to KIAA1387 | |
| | | protein (H. sapiens) | |
| 25.16667 | 11.55556 | Homo sapiens full length | Yes |
| | | insert cDNA clone YW25E05 | |
| 24.83333 | 29.83333 | Hs.208486 /UG_TITLE = ESTs | |
| 24.7619 | 11.90476 | Homo sapiens mRNA; cDNA | |
| | | DKFZp313L0839 (from clone | |
| | | DKFZp313L0839). | |
| 24.71429 | 14.92857 | Homo sapiens synaptonemal | |
| | | complex protein 1 (SYCP1), | |
| | | mRNA. /PROD = synaptonemal | |
| | | complex protein 1 | |
| | | /FL = gb: NM_003176.1 gb: D67 | |
| 24.64286 | 2.785714 | Homo sapiens cDNA FLJ14020 | |
| | | fis, clone HEMBA1002508. | |
| 24.09091 | 27.18182 | Homo sapiens, clone | |
| | | IMAGE: 5269594, mRNA. | |
| 23.73333 | 1.933333 | Hs.99578 /UG_TITLE = ESTs, | Yes |
| | | Highly similar to | |
| | | PTPD_HUMAN PROTEIN- | |
| | | TYROSINE PHOSPHATASE | |
| | | DELTA PRECURSOR | |
| | | (H. sapiens) | |
| 23.58065 | 22.03226 | H. sapiens mRNA for | |
| | | gonadotropin-releasing | |
| | | hormone receptor, splice | |
| | | variant. /PROD = gonadotropin- | |
| | | releasing | |
| | | hormone receptor | |
| 23.3913 | 4.913043 | Homo sapiens cDNA FLJ12548 | Yes |
| | | fis, clone NT2RM4000657, | |
| | | weakly similar to 1- | |
| | | PHOSPHATIDYLINOSITO | |
| 23 | 2.833333 | Homo sapiens cDNA FLJ37910 | Yes |
| | | fis, clone CTONG1000040. | |
| 22.65 | 17.15 | Homo sapiens mRNA for pH- | |
| | | sensing regulatory factor | |
| | | of peptide transporter, | |
| | | complete cds. | |
| 22.35714 | 2 | Homo sapiens, clone | Yes |
| | | IMAGE: 4398590, mRNA. | |
| 22.125 | 0.625 | Homo sapiens cDNA: FLJ20870 | Yes |
| | | fis, clone ADKA02524. | |
| 21.83333 | 13.58333 | Homo sapiens cDNA FLJ11096 | |
| | | fis, clone PLACE1005480. | |
| 21.8 | 7.1 | Hs.60556 /UG_TITLE = ESTs | Yes |
| 21.47059 | 1.058824 | Homo sapiens, clone | Yes |
| | | IMAGE: 5742085, mRNA. | |
| 21.4 | 1.2 | Hs.130544 /UG_TITLE = ESTs | Yes |
| 21 | 7.208333 | Hs.222222 /UG_TITLE = ESTs | Yes |
| 20.85714 | 7.214286 | Homo sapiens osteoglycin | Yes |
| | | (osteoinductive factor, | |
| | | mimecan) (OGN), mRNA | |
| 20.73913 | 17.82609 | Hs.222120 /UG_TITLE = ESTs | |
| 20.71429 | 19.78571 | Homo sapiens cDNA FLJ25595 | |
| | | fis, clone JTH13269. | |
| 20.7 | 4.3 | Homo sapiens cDNA FLJ13003 | Yes |
| | | fis, clone NT2RP3000418. | |
| 20.42857 | 7.035714 | Human DNA sequence from | Yes |
| | | clone 733D15 on chromosome | |
| | | Xp11.3. Contains a Zinc- | |
| | | finger (pseudo?) gene and G | |
| 20.42857 | 6.714286 | Hs.132649 /UG_TITLE = ESTs | Yes |
| 20.28235 | 10.15294 | Homo sapiens cDNA FLJ11475 | Yes |
| | | fis, clone HEMBA1001734, | |
| | | moderately similar to | |
| | | CADHERIN-11 PRECURSOR | |
| 20.25 | 4.75 | Homo sapiens epiregulin | Yes |
| | | (EREG), mRNA | |
| 20.2381 | 15.52381 | Homo sapiens cDNA FLJ39700 | |
| | | fis, clone SMINT2011588, | |
| | | weakly similar to Kruppe | |
| 20.04545 | 9.136364 | Homo sapiens mRNA; cDNA | Yes |
| | | DKFZp434P2450 (from clone | |
| | | DKFZp434P2450). | |
| 19.95238 | 1 | paraneoplastic | Yes |
| | | encephalomyelitis antigen | |
| | | {5 region, alternatively | |
| | | spliced} (human, lung | |
| | | cancer cell line, mRNA | |
| | | Partial, 10 | |
| 19.78261 | 15.65217 | Homo sapiens aldehyde | |
| | | oxidase 1 (AOX1), mRNA | |
| 19.77778 | 13.77778 | Hs.293582 /UG_TITLE = ESTs | |
| 19.75 | 6.833333 | Homo sapiens cDNA: FLJ21221 | Yes |
| | | fis, clone COL00570. | |
| 19.71795 | 15.89744 | Homo sapiens cDNA FLJ40624 | |
| | | fis, clone THYMU2013981. | |
| 19.69565 | 23.95652 | colony stimulating factor 2 | |
| | | receptor, beta, low- | |
| | | affinity (granulocyte- | |
| | | macrophage) | |
| 19.6 | 12.46667 | Human deleted in | |
| | | azoospermia protein (DAZ) | |
| | | mRNA, complete cds | |
| 19.57143 | 19.85714 | glutamate receptor, | |
| | | ionotropic, kainate 2 | |
| | | /DEF = Homo sapiens mRNA for | |
| | | GluR6 kainate receptor | |
| | | (GRIK2 gene), isoform-b | |
| 19.55556 | 2.111111 | hypothetical protein | Yes |
| | | LOC285419 /DEF = Homo | |
| | | sapiens, clone | |
| | | IMAGE: 4839001, mRNA | |
| 19.52941 | 1.235294 | Homo sapiens sperm | Yes |
| | | associated antigen 11 | |
| | | (SPAG11), transcript | |
| | | variant B, mRNA. | |
| 19.25926 | 9.185185 | Homo sapiens mRNA; cDNA | Yes |
| | | DKFZp434L1717 (from clone | |
| | | DKFZp434L1717); complete | |
| | | cds | |
| 19.24138 | 20.31034 | Homo sapiens cDNA FLJ35054 | |
| | | fis, clone OCBBF2018380. | |
| 19.16667 | 14.11111 | Hs.36683 /UG_TITLE = ESTs | |
| 19.07143 | 8 | Hs.106645 /UG_TITLE = ESTs | Yes |
| 18.93333 | 37.53333 | Homo sapiens cDNA FLJ11602 | |
| | | fis, clone HEMBA1003908 | |
| 18.90476 | 15.38095 | Homo sapiens, clone | |
| | | IMAGE: 5164933, mRNA | |
| 18.71429 | 40.71429 | Hs.176420 /UG_TITLE = ESTs | |
| 18.7037 | 2.222222 | Homo sapiens, Similar to | Yes |
| | | BCL2-associated athanogene, | |
| | | clone IMAGE: 4310445, mRNA | |
| 18.66667 | 20.06667 | DNA segment on chromosome X | |
| | | (unique) 9928 expressed | |
| | | sequence | |
| 18.5 | 5.958333 | Homo sapiens PIAS-NY | Yes |
| | | protein mRNA, complete cds | |
| 18.47368 | 4.368421 | Homo sapiens full length | Yes |
| | | insert cDNA clone YI41H11 | |
| 18.46154 | 4.615385 | Homo sapiens pre-TNK cell | Yes |
| | | associated protein (1D12A), | |
| | | mRNA | |
| 18.45455 | 17.24242 | Homo sapiens mRNA | |
| | | differentially expressed in | |
| | | malignant melanoma, clone | |
| F | | MM K2 | |
| 18.23529 | 3.529412 | Homo sapiens cDNA FLJ32062 | Yes |
| | | fis, clone OCBBF1000042. | |
| 18.14286 | 6 | Hs.204562 /UG_TITLE = ESTs | Yes |
| 18.03226 | 7.451613 | Hs.269931 /UG_TITLE = ESTs | Yes |
| 18 | 13.58333 | Homo sapiens, clone | |
| | | IMAGE: 4393885, rnRNA, | |
| | | partial cds | |
| 17.76471 | 24.88235 | Hs.23187 /UG_TITLE = ESTs | |
| 17.71429 | 23.07143 | Hs.42993 /UG_TITLE = ESTs | |
| 17.625 | 1.5 | Homo sapiens glypican 5 | Yes |
| | | (GPC5), mRNA | |
| 17.6129 | 12.12903 | Homo sapiens mRNA; cDNA | |
| | | DKFZp686C1636 (from clone | |
| | | DKFZp686C1636) | |
| 17.5 | 21.53125 | Hs.213386 /UG_TITLE = ESTs | |
| 17.48276 | 2 | Hs.99200 /UG_TITLE = ESTs | Yes |
| 17.47826 | 2.913043 | Hs.17388 /UG_TITLE = ESTs | Yes |
| 17.47368 | 6.710526 | MCF.2 cell line derived | Yes |
| | | transforming sequence-like | |
| | | /DEF = Homo sapiens, clone | |
| | | IMAGE: 5185971, mRNA | |
| 17.40541 | 3.486486 | Hs.6656 /UG_TITLE = ESTs | Yes |
| 17.36842 | 15.73684 | Hs.22249 /UG_TITLE = ESTs | |
| 17.31169 | 16.42857 | Hs.20103 /UG_TITLE = ESTs | |
| 17.09091 | 17.09091 | Human (clone CTG-A4) mRNA | |
| | | sequence | |
| 17.09091 | 22.36364 | Homo sapiens cDNA FLJ36285 | |
| | | fis, clone THYMU2003470. | |
| 17 | 16.44444 | Homo sapiens SAM domain, | |
| | | SH3 domain and nuclear | |
| | | localisation signals, 1 | |
| | | (SAMSN1), mRNA. /PROD = SAM | |
| | | domain, SH3 domain and | |
| | | nuclear | |
| 16.83333 | 17.69444 | Homo sapiens, clone | |
| | | MGC: 34025 IMAGE: 4828588, | |
| | | mRNA, complete cds. | |
| | | /PROD = Unknown (protein for | |
| | | MGC: 34025) | |
| 16.83333 | 15.58333 | Homo sapiens, clone | |
| | | IMAGE: 4838843, mRNA | |
| 16.81818 | 0.727273 | Hs.97977 /UG_TITLE = ESTs | Yes |
| 16.75556 | 4.377778 | Homo sapiens mRNA; cDNA | Yes |
| | | DKFZp586O2023 (from clone | |
| | | DKFZp586O2023) | |
| 16.66667 | 17.75 | Hs.36409.0 /TIER = ConsEnd | |
| | | /STK = 4 /UG = Hs.36409 | |
| | | /UG_TITLE = ESTs | |
| 16.65789 | 11.65789 | Human hepatocyte nuclear | |
| | | factor-6 alpha (HNF6) mRNA, | |
| | | complete cds | |
| 16.625 | 3.5 | Hs.188950 /UG_TITLE = ESTs | Yes |
| 16.625 | 11.0625 | Hs.277419 /UG_TITLE = ESTs | |
| 16.61111 | 12.77778 | pancreatic ribonuclease | |
| | | (human, mRNA Recombinant | |
| | | Partial, 491 nt) | |
| 16.54545 | 15.09091 | Homo sapiens, clone | |
| | | IMAGE: 5277680, mRNA, | |
| | | partial cds. | |
| 16.53488 | 3.860465 | Homo sapiens glutamate | Yes |
| | | receptor, ionotrophic, AMPA | |
| | | 4 (GRIA4), mRNA. | |
| | | /PROD = glutamate receptor, | |
| | | ionotrophic /FL = gb: U16129.1 | |
| | | gb: NM_0 | |
| 16.46575 | 11.61644 | endothelin receptor type A | |
| | | /FL = gb: NM_001957.1 | |
| | | gb: L06622.1 | |
| 16.42857 | 4.761905 | Hs.99472 /UG_TITLE = ESTs | Yes |
| 16.42105 | 5.631579 | Homo sapiens mRNA for type | Yes |
| | | I keratin. /PROD = HHa5 hair | |
| | | keratin type I intermediate | |
| | | filament | |
| 16.40909 | 3.227273 | Homo sapiens protein | Yes |
| | | tyrosine phosphatase, | |
| | | receptor-type, Z | |
| | | polypeptide 1 (PTPRZ1), | |
| | | mRNA | |
| 16.36 | 22.4 | Homo sapiens neuropeptide Y | |
| | | receptor Y5 (NPY5R), mRNA | |
| 16.2 | 48.8 | Homo sapiens cDNA: FLJ20890 | |
| | | fis, clone ADKA03323. | |
| 16.05882 | 3.882353 | syntaphilin | Yes |
| 16 | 13.5 | Human DNA sequence from | |
| | | clone RP4-545L17 on | |
| | | chromosome 20p12.2-13. | |
| | | Contains the 5 end of the | |
| | | gene for a novel protein | |
| | | similar to RAD21 (S. pom | |
| 15.95522 | 8.552239 | Homo sapiens cDNA FLJ36177 | Yes |
| | | fis, clone TESTI2026515. | |
| 15.91667 | 0.5 | Hs.40840 /UG_TITLE = ESTs | Yes |
| 15.89474 | 25.12281 | Homo sapiens cDNA FLJ13602 | |
| | | fis, clone PLACE1010089, | |
| | | highly similar to Homo | |
| | | sapiens mRNA for | |
| 15.88235 | 1.156863 | Human CB-4 transcript of | Yes |
| | | unrearranged immunoglobulin | |
| | | V(H)5 gene /DEF = Human CLL- | |
| | | 12 transcript of | |
| | | unrearranged immuno | |
| 15.875 | 18.5 | Homo sapiens, clone | |
| | | IMAGE: 4823434, mRNA | |
| 15.85185 | 1.888889 | Hs.173596 /UG_TITLE = ESTs | Yes |
| 15.83333 | 3.944444 | Homo sapiens GPBP- | Yes |
| | | interacting protein 90 | |
| | | mRNA, complete cds | |
| 15.81481 | 0.888889 | Homo sapiens, Similar to | Yes |
| | | recombination activating | |
| | | gene 1, clone MGC: 43321 | |
| | | IMAGE: 5265661, mRNA, | |
| | | complete cds | |
| 15.72414 | 2.241379 | Homo sapiens cDNA FLJ37001 | Yes |
| | | fis, clone BRACE2008172. | |
| 15.71429 | 11.42857 | Homo sapiens, Similar to | |
| | | RIKEN cDNA 4833427G06 gene, | |
| | | clone IMAGE: 5561932, mRNA | |
| 15.6875 | 9.0625 | Homo sapiens, clone | |
| | | IMAGE: 4831108, mRNA | |
| 15.6875 | 0.78125 | Homo sapiens, clone | Yes |
| | | IMAGE: 5295305, mRNA | |
| 15.63636 | 5.681818 | Hs.98388 /UG_TITLE = ESTs | Yes |
| 15.61538 | 11.11538 | methyl-CpG binding domain | |
| | | protein 2 | |
| 15.59677 | 267.7419 | Hs.154993 /UG_TITLE = ESTs | |
| 15.54545 | 9.181818 | Homo sapiens transmembrane | |
| | | phosphatase with tensin | |
| | | homology (TPTE), mRNA. | |
| 15.52632 | 10.31579 | Hs.105620 /UG_TITLE = ESTs | |
| 15.42857 | 23.28571 | Homo sapiens acetyl LDL | |
| | | receptor; SREC = scavenger | |
| | | receptor expressed by | |
| | | endothelial cells (SREC), | |
| | | mRNA. /PROD = acetyl LDL | |
| | | receptor; SR | |
| 15.36842 | 0.684211 | Homo sapiens cDNA: FLJ21351 | Yes |
| | | fis, clone COL02762 | |
| 15.36364 | 1.454545 | Homo sapiens cDNA FLJ30168 | Yes |
| | | fis, clone BRACE2000750. | |
| 15.3 | 12.83333 | Homo sapiens clone 148022 | |
| | | iduronate-2-sulfatase | |
| | | (IDS2) pseudogene, mRNA | |
| | | sequence | |
| 15.27027 | 3.324324 | Homo sapiens microtubule- | Yes |
| | | associated protein tau | |
| | | (MAPT), transcript variant | |
| | | 1, mRNA. | |
| 15.23077 | 12.15385 | Homo sapiens, clone | |
| | | IMAGE: 4830182, mRNA. | |
| 15.21429 | 2.642857 | Homo sapiens mRNA; cDNA | Yes |
| | | DKFZp434H0872 (from clone | |
| | | DKFZp434H0872). | |
| 15.21053 | 1.684211 | Homo sapiens cDNA: FLJ22630 | Yes |
| | | fis, clone HSI06250. | |
| 15.19231 | 11.76923 | Homo sapiens H2B histone | |
| | | family, member N (H2BFN), | |
| | | mRNA | |
| 15.19048 | 1.47619 | Homo sapiens cDNA FLJ11921 | Yes |
| | | fis, clone HEMBB1000318. | |
| 15.16667 | 1.233333 | Hs.168268 /UG_TITLE = ESTs, | Yes |
| | | Moderately similar to | |
| | | A35969 heparin-binding | |
| | | growth factor receptor K- | |
| | | sam precursor (H. sapiens) | |
| 15.125 | 1.1875 | Human EST clone 53125 | Yes |
| | | mariner transposon Hsmar1 | |
| | | sequence | |
| 15.09375 | 4 | Homo sapiens, clone | Yes |
| | | MGC: 47837 IMAGE: 6046539, | |
| | | mRNA, complete cds. | |
| | | /PROD = Unknown (protein for | |
| | | MGC: 47837) | |
| 15.07407 | 4.277778 | Homo sapiens cDNA: FLJ21710 | Yes |
| | | fis, clone COL10087. | |
| 15.06667 | 15.96667 | Hs.143834 /UG_TITLE = ESTs | |
| 15.05128 | 5.512821 | hypothetical protein | Yes |
| | | FLJ20271 /FL = gb: NM_017734.1 | |
| 15.02564 | 5.974359 | Homo sapiens full length | Yes |
| | | insert cDNA clone YP60H04 | |
| 15.02326 | 11.44186 | Homo sapiens calsyntenin-2 | |
| | | (CS2), mRNA. | |
| | | /PROD = calsyntenin-2 | |
| 15 | 1.5 | Hs.104572 /UG_TITLE = ESTs | Yes |
| 14.9403 | 12.67164 | Homo sapiens inhibin, beta | |
| | | C (INHBC), mRNA. | |
| | | /PROD = inhibin beta C | |
| | | subunit precursor | |
| 14.88462 | 11.82692 | Homo sapiens cDNA FLJ10146 | |
| | | fis, clone HEMBA1003327 | |
| 14.80851 | 15.65957 | gb: AW451826 | |
| | | /DB_XREF = gi: 6992602 | |
| | | /DB_XREF = UI-H-BI3-alk-e-07- | |
| | | 0-UI.s1 | |
| | | /CLONE = IMAGE: 2737236 | |
| | | /FEA = EST /CNT = 8 | |
| | | /TID = Hs.258791.0 | |
| | | /TIER = ConsEnd /STK = 4 | |
| | | /UG = Hs.258791 | |
| | | /UG_TITLE = ESTs | |
| 14.78788 | 5.348485 | gb: BF590323 | Yes |
| | | /DB_XREF = gi: 11682647 | |
| | | /DB_XREF = nab22h10.x1 | |
| | | /CLONE = IMAGE: 3266922 | |
| | | /FEA = EST /CNT = 33 | |
| | | /TID = Hs.55256.0 /TIER = Stack | |
| | | /STK = 30 /UG = Hs.55256 | |
| | | /UG_TITLE = ESTs | |
| 14.71154 | 0.442308 | Homo sapiens, clone | Yes |
| | | IMAGE: 4815474, mRNA | |
| 14.69565 | 12.67391 | Homo sapiens RAGE mRNA for | |
| | | advanced glycation | |
| | | endproducts receptor, | |
| | | complete cds. | |
| 14.65217 | 22.82609 | Homo sapiens, clone | |
| | | MGC: 10724, mRNA, complete | |
| | | cds. /PROD = Unknown (protein | |
| | | for MGC: 10724) | |
| 14.64286 | 2.571429 | Homo sapiens mRNA; cDNA | Yes |
| | | DKFZp761D191 (from clone | |
| | | DKFZp761D191) | |
| 14.61538 | 1.307692 | Hs.313876 /UG_TITLE = ESTs | Yes |
| 14.53488 | 8.348837 | CGI-67 protein | |
| 14.5 | 9.772727 | Hs.132650 /UG_TITLE = ESTs | |
| 14.42857 | 1.619048 | Hs.293685 /UG_TITLE = ESTs | Yes |
| 14.41667 | 4.708333 | Hs.143789 /UG_TITLE = ESTs | Yes |
| 14.40909 | 5.181818 | Homo sapiens cDNA FLJ13755 | Yes |
| | | fis, clone PLACE3000363. | |
| 14.33333 | 2.888889 | Hs.327117 /UG_TITLE = ESTs | Yes |
| 14.29032 | 1.903226 | Hs.161566 /UG_TITLE = ESTs | Yes |
| 14.27778 | 1.055556 | Homo sapiens, clone | Yes |
| | | IMAGE: 4778480, mRNA. | |
| 14.23077 | 15.84615 | Homo sapiens, Similar to | |
| | | hypothetical protein | |
| | | FLJ22792, clone MGC: 22933 | |
| | | IMAGE: 4905554, mRNA, | |
| | | complete cds | |
| 14.20513 | 1 | Hs.162565 /UG_TITLE = ESTs | Yes |
| 14.14815 | 21.37037 | Homo sapiens, Similar to | |
| | | sex comb on midleg-like 3 | |
| | | (Drosophila), clone | |
| | | MGC: 25118 IMAGE: 4509724, | |
| | | mRNA, complete cds. | |
| 14.14286 | 11.57143 | Homo sapiens olfactory-like | |
| | | receptor JCG8 (JCG8) mRNA, | |
| | | complete cds. | |
| | | /PROD = olfactory-like | |
| | | receptor JCG8 | |
| 14.1 1 | 5.3 | Homo sapiens, clone | |
| | | IMAGE: 5267701, mRNA | |
| 14.06667 | 5.15 | Homo sapiens cDNA FLJ34667 | Yes |
| | | fis, clone LIVER2000769. | |
| | | /DEF = Homo sapiens cDNA | |
| | | FLJ34667 fis, clone | |
| | | LIVER2000769. | |
| 14.05882 | 7.588235 | major histocompatibility | Yes |
| | | complex, class II, DR beta 3 | |
| 14.05882 | 3.176471 | Hs.125962 /UG_TITLE = ESTs | Yes |
| 14.05833 | 6.35 | Hs.293118 /UG_TITLE = ESTs | Yes |
| 14.03896 | 13.66234 | Hs.20726 /UG_TITLE = ESTs | |
| 14.02778 | 9.555556 | Homo sapiens, clone | |
| | | MGC: 14510, mRNA, complete | |
| | | cds. /PROD = Unknown (protein | |
| | | for MGC: 14510) | |
| 14.02632 | 17.39474 | Homo sapiens CD84 antigen | |
| | | (leukocyte antigen) (CD84), | |
| | | mRNA. /PROD = CD84 antigen | |
| | | (leukocyte antigen) | |
| 14 | 11.90909 | Hs.296235 /UG_TITLE = ESTs | |
| 14 | 38.29412 | prostate specific G-protein | |
| | | coupled receptor /DEF = Homo | |
| | | sapiens prostate specific | |
| | | G-protein coupled receptor | |
| | | gene, comple | |
| 14 | 22.2 | Homo sapiens cystic | |
| | | fibrosis transmembrane | |
| | | conductance regulator | |
| | | isoform 36 (CFTR) mRNA, | |
| | | partial cds | |
| 13.97826 | 12.54348 | Homo sapiens testis | |
| | | transcript Y 9 (TTY9) mRNA, | |
| | | complete cds | |
| 13.90625 | 0.9375 | Hs.88450 /UG_TITLE = ESTs | Yes |
| 13.9 | 1.1 | Hs.20468 /UG_TITLE = ESTs | Yes |
| 13.89474 | 3.421053 | Homo sapiens cDNA: FLJ21618 | Yes |
| | | fis, clone COL07487. | |
| 13.85294 | 2.352941 | Homo sapiens fibroblast | Yes |
| | | growth factor 20 (FGF20), | |
| | | mRNA | |
| 13.81818 | 3.136364 | Homo sapiens mRNA; cDNA | Yes |
| | | DKFZp761J1323 (from clone | |
| | | DKFZp761J1323). | |
| 13.80769 | 2.307692 | Hs.407438 | Yes |
| | | /UG_TITLE = neurogenic | |
| | | differentiation 1 | |
| 13.78788 | 12.45455 | Homo sapiens hypothetical | |
| | | protein FLJ12983 | |
| | | (FLJ12983), mRNA | |
| 13.7549 | 9.77451 | Human DNA sequence from | |
| | | clone RP5-1184F4 on | |
| | | chromosome 20q11.1-11.23. | |
| | | Contains the 3 end of gene | |
| | | KIAA0978, two genes for | |
| | | novel proteins similar | |
| 13.73333 | 8.8 | Hs.201420 /UG_TITLE = ESTs | |
| 13.71429 | 12.17857 | Homo sapiens cDNA FLJ12573 | |
| | | fis, clone NT2RM4000979 | |
| 13.7 | 23.3 | Hs.244710 /UG_TITLE = ESTs | |
| 13.68293 | 5.585366 | Homo sapiens tenascin R | Yes |
| | | (restrictin, janusin) | |
| | | (TNR), mRNA. /PROD = tenascin | |
| | | R (restrictin, janusin) | |
| 13.66667 | 1.606061 | Hs.99336 /UG_TITLE = ESTs | Yes |
| 13.64045 | 9.280899 | Homo sapiens testis- | |
| | | specific ankyrin motif | |
| | | containing protein | |
| | | (LOC56311), mRNA. | |
| 13.61538 | 1.846154 | Hs.130922 /UG_TITLE = Homo | Yes |
| | | sapiens, Similar to likely | |
| | | ortholog of yeast ARV1, | |
| | | clone IMAGE: 5265646, mRNA | |
| 13.59259 | 0.814815 | olfactory receptor, family | Yes |
| | | 2, subfamily M, member 4 | |
| | | /DEF = H. sapiens mRNA for | |
| | | TPCR100 protein. | |
| 13.57143 | 3.142857 | Homo sapiens, clone | Yes |
| | | IMAGE: 4694422, mRNA. | |
| 13.55882 | 3.661765 | Homo sapiens small | Yes |
| | | intestine aquaporin mRNA, | |
| | | complete cds | |
| 13.55172 | 3.689655 | Homo sapiens mRNA; cDNA | Yes |
| | | DKFZp564I083 (from clone | |
| | | DKFZp564I083) | |
| 13.55172 | 1.448276 | gb: H47594 | Yes |
| | | /DB_XREF = gi: 923646 | |
| | | /DB_XREF = yp75c01.s1 | |
| | | /CLONE = IMAGE: 193248 | |
| | | /TID = Hs2.407314.1 /CNT = 3 | |
| | | /FEA = mRNA /TER = ConsEnd | |
| | | /STK = 1 /UG = Hs.407314 | |
| | | /UG_TITLE = Homo sapiens full | |
| | | length insert cDNA clone | |
| | | YP75C01 | |
| 13.53846 | 7.807692 | Homo sapiens cDNA FLJ39005 | |
| | | fis, clone NT2RI2024496 | |
| 13.52941 | 11.2549 | gb: H46217 | |
| | | /DB_XREF = gi: 922269 | |
| | | /DB_XREF = yo14h12.s1 | |
| | | /CLONE = IMAGE: 177959 | |
| | | /FEA = EST /CNT = 4 | |
| | | /TID = Hs.268805.0 | |
| | | /TIER = ConsEnd /STK = 4 | |
| | | /UG = Hs.268805 | |
| | | /UG_TITLE = ESTs | |
| 13.5 | 8.535714 | Hs.250113 /UG_TITLE = ESTs, | |
| | | Moderately similar to | |
| | | thyroid hormone receptor- | |
| | | associated protein complex | |
| | | component TRAP150 (H. sap | |
| 13.40909 | 9.863636 | Homo sapiens, clone | |
| | | IMAGE: 3933453, mRNA | |
| 13.36842 | 0.842105 | Hs.28714 /UG_TITLE = ESTs | Yes |
| 13.35714 | 1.357143 | Homo sapiens, clone | Yes |
| | | IMAGE: 5266862, mRNA. | |
| 13.35135 | 12.81081 | Hs.158937 /UG_TITLE = ESTs | |
| 13.35 | 1.65 | Homo sapiens cDNA FLJ13136 | Yes |
| | | fis, clone NT2RP3003139 | |
| 13.33333 | 6.6 | Homo sapiens non-coding RNA | Yes |
| | | HANC | |
| 13.30769 | 7.410256 | Hs.25046 /UG_TITLE = ESTs | |
| 13.29384 | 8.21327 | Homo sapiens protein kinase | |
| | | C, alpha binding protein | |
| | | (PRKCABP), mRNA | |
| 13.2807 | 4.017544 | Homo sapiens hypothetical | Yes |
| | | protein FLJ10979 | |
| | | (FLJ10979), mRNA. | |
| | | /PROD = hypothetical protein | |
| | | FLJ10979 | |
| 13.25 | 7.15 | Homo sapiens full length | |
| | | insert cDNA clone YI41B09 | |
| 13.24138 | 0.896552 | Homo sapiens, clone | Yes |
| | | IMAGE: 4818264, mRNA | |
| 13.2381 | 10.2619 | Homo sapiens, clone | |
| | | IMAGE: 4824978, mRNA | |
| 13.23333 | 15.1 | gb: AA776626 | |
| | | /DB_XREF = gi: 2835960 | |
| | | /DB_XREF = ae86f02.s1 | |
| | | /CLONE = IMAGE: 971067 | |
| | | /FEA = EST /CNT = 12 | |
| | | /TID = Hs.62183.0 | |
| | | /TIER = ConsEnd /STK = 1 | |
| | | /UG = Hs.62183 /UG_TITLE = | |
| | | ESTs | |
| 13.2 | 8 | myelin oligodendrocyte | |
| | | glycoprotein /DEF = Human DNA | |
| | | sequence from clone RP11- | |
| | | 145L22 on chromosome | |
| | | 6p21.32-22.2 | |
| 13.18182 | 0.818182 | Homo sapiens regulator of | Yes |
| | | G-protein signaling 1 | |
| | | (RGS1), mRNA. | |
| | | /PROD = regulator of G- | |
| | | protein signaling 1 | |
| 13.11111 | 12.11111 | Homo sapiens clone HQ0202 | |
| | | PRO0202 mRNA, partial cds | |
| 13.09091 | 17.30303 | cytoplasmic linker | |
| | | associated protein 2 | |
| 13.09091 | 17.63636 | H. sapiens AA1 mRNA | |
| 13.04762 | 1.380952 | Homo sapiens, clone | Yes |
| | | IMAGE: 4825614, mRNA. | |
| 13 | 1.75 | Human clone 23909 mRNA, | Yes |
| | | partial cds. /PROD = unknown | |
| 13 | 1.657143 | Homo sapiens cDNA FLJ12289 | Yes |
| | | fis, clone MAMMA1001788 | |
| 12.93333 | 11.66667 | Homo sapiens mRNA for | |
| | | keratin associated protein | |
| | | 4.7 (KRTAP4.7 gene) | |
| 12.93103 | 0.413793 | Hs.43052 /UG_TITLE = ESTs | Yes |
| 12.92308 | 4.846154 | Homo sapiens cDNA FLJ14152 | Yes |
| | | fis, clone MAMMA1003089. | |
| 12.88 | 1.52 | Hs.118342 /UG_TITLE = ESTs | Yes |
| 12.86207 | 6.965517 | Homo sapiens, clone | |
| | | IMAGE: 4042783, mRNA. | |
| 12.84 | 11.68 | Homo sapiens POU domain, | |
| | | class 4, transcription | |
| | | factor 2 (POU4F2), mRNA. | |
| | | /PROD = POU domain, class 4, | |
| | | transcription factor 2 | |
| 12.83333 | 8.111111 | Hs.190319 /UG_TITLE = ESTs | |
| 12.8 | 3.72 | Hs.231951 /UG_TITLE = ESTs | Yes |
| 12.8 | 9.766667 | Homo sapiens olfactory | |
| | | receptor-like protein JCG3 | |
| | | (JCG3), mRNA | |
| 12.78947 | 1.263158 | Homo sapiens, clone | Yes |
| | | IMAGE: 4413783, mRNA. | |
| 12.78788 | 1.181818 | Homo sapiens, clone | Yes |
| | | IMAGE: 4800001, mRNA. | |
| 12.76923 | 2 | Homo sapiens, clone | Yes |
| | | IMAGE: 4828930, mRNA. | |
| 12.76471 | 1.705882 | Homo sapiens mRNA expressed | Yes |
| | | only in placental villi, | |
| | | clone SMAP41 | |
| 12.75 | 7.08333 | Hs.259168 /UG_TITLE = ESTs | |
| 12.69565 | 17.47826 | Homo sapiens hypothetical | |
| | | protein FLJ21272 | |
| | | (FLJ21272), mRNA | |
| 12.6875 | 7.333333 | Hs.92955 /UG_TITLE = ESTs | |
| 12.67857 | 4.071429 | hypothetical protein | Yes |
| | | FLJ10024 /DEF = Homo sapiens | |
| | | cDNA FLJ13978 fis, clone | |
| | | Y79AA1001665. | |
| 12.66102 | 9.050847 | Homo sapiens RNA binding | |
| | | motif protein, Y | |
| | | chromosome, family 2, | |
| | | member B (RBMY2B) mRNA. | |
| | | /PROD = RNA binding motif | |
| | | protein, Y chromos | |
| 12.61538 | 1.384615 | Hs.127556 /UG_TITLE = ESTs | Yes |
| 12.57576 | 9.636364 | Hs.44736 /UG_TITLE = ESTs | |
| 12.55172 | 0.724138 | hypothetical protein | Yes |
| | | LOC285965 /DEF = Homo sapiens | |
| | | mRNA; cDNA DKFZp686O0656 | |
| | | (from clone DKFZp686O0656). | |
| 12.54839 | 7.709677 | Hs.276363 | |
| | | /UG_TITLE = hypothetical | |
| | | protein LOC283112 | |
| 12.53333 | 14.86667 | REPL1S /UG_TITLE = ret finger | |
| | | protein-like 1 antisense | |
| 12.50847 | 1.559322 | Hs.98945 /UG_TITLE = ESTs | Yes |
| 12.5 | 6.136364 | Hs.213371 /UG_TITLE = ESTs | Yes |
| 12.45946 | 7.297297 | Homo sapiens, similar to | |
| | | hypothetical protein, clone | |
| | | MGC: 27103 IMAGE: 4831323, | |
| | | mRNA, complete cds. | |
| 12.42424 | 16.54545 | Homo sapiens GLB2 gene, | |
| | | upstream regulatory region | |

With respect to the potential role of HTLV-1 p30^{II} in adult T-cell leukemogenesis, transcriptional activation of the following genes is of significant interest: myeloid cell nuclear differentiation 1 antigen (31.1-fold; TIP60-dependent), protocadherin 15 (26.1-fold; TIP60-dependent), human protein tyrosine-phosphatase delta precursor (23.3-fold; TIP60-dependent), cadherin 11-like precursor (20.2-fold; TIP60-dependent), colony stimulating factor 2 receptor, beta (19.6-fold; TIP60-independent), human protein tyrosine-phosphatase receptor-type Z polypeptide (16.4-fold; TIP60-dependent), S. pombe RAD21-like protein (16-fold; TIP60-independent), human transmembrane phosphatase with tensin homology (15.5-fold; TIP60-independent), H2B histone family member N (15.1-fold; TIP60-independent), major histocompatibility complex class II DR beta 3 (14.0-fold; TIP60-dependent), human CD84 leukocyte antigen (14.0-fold; TIP60-independent), prostate-specific G-protein coupled receptor (14.0-fold; TIP60-independent), fibroblast growth factor 20 (13.8-fold; TIP60-dependent), protein kinase C alpha-binding protein (13.2-fold; TIP60-independent), regulator of G-protein-signaling 1 (13.1-fold; TIP60-dependent), cytoplasmic linker associated protein 2 (13.0-fold; TIP60-independent), POU domain 4 transcription factor 2 (12.8-fold; TIP60-independent), and RNA-binding motif protein (RBMY2B) (12.6-fold; TIP60-independent). Infectious HTLV-1 molecular clone, ACH.p30^{II}, exhibits an approx 20-50% reduction in transformation-efficiency compared to the wild-type ACH.wt suggesting that p30^{II} is required for the full-transforming potential of HTLV-1. Microarray analyses indicates that numerous cellular genes are transcriptionally activated by p30^{II}, and proteins encoded by these genes may contribute to HTLV-1 leukemic transformation and development of ATLL.

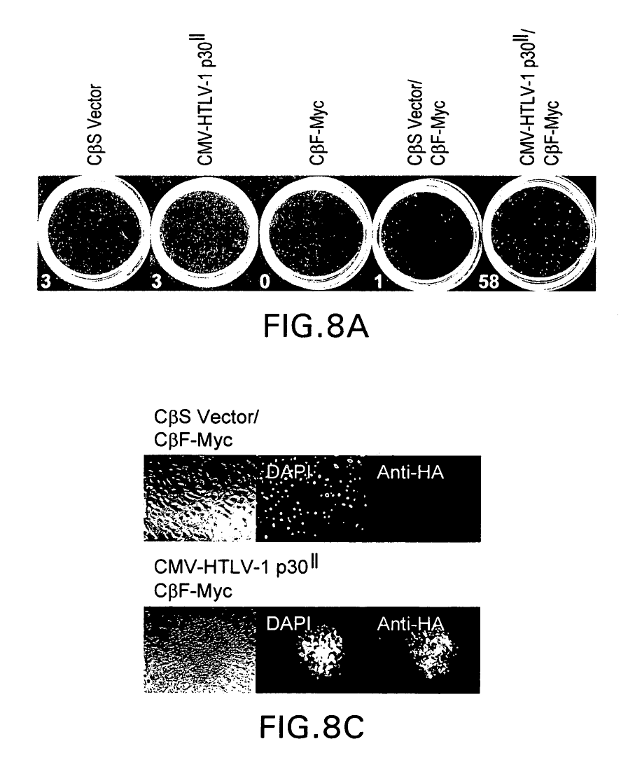

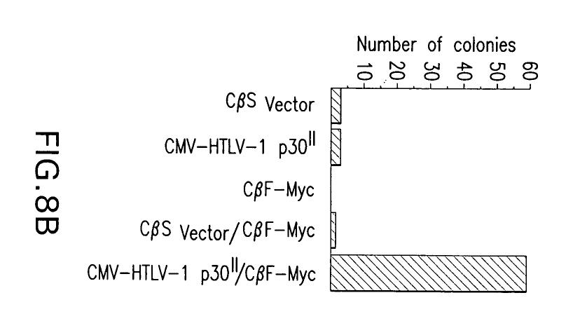

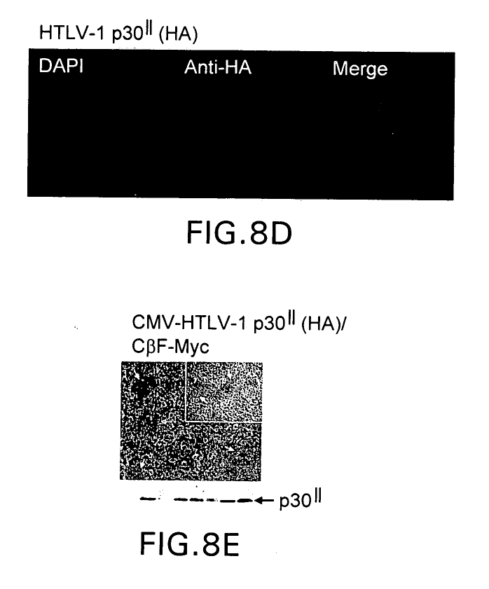

HTLV-1 p30^{II} enhances Myc transforming potential and requires the TIP60 HAT and TRRAP/p434. Since the c-Myc oncogene is known to cause cellular transformation, foci-formation assays using immortalized human WRN^{-/-} fibroblasts, which lack Werner's Syndrome helicase functions were used to determine whether HTLV-1 p30^{II} might influence Myc-associated transforming activity. This cellular background was chosen because ATLL is an aging-related malignancy requiring clinical latency periods of 25-40 years prior to disease onset, which suggests that genetic mutations linked to the aging process likely contribute to leukemogenesis. Werner's Syndrome is a premature-aging disorder that mimics or recapitulates many of the clinical and cellular features of normal aging; and WRN locus (8p11-12) mutations have been found in HTLV-1-infected ATLL patient lymphocytes and in HTLV-1-infected mycosis fungoides/Sezary syndrome cells. Neither Myc nor HTLV-1 p30^{II} (HA) alone significantly induces foci-formation in immortalized human WRN^{-/-} fibroblasts (FIG. 8A). Surprisingly, in combination, HTLV-1 p30^{II} (HA)-Myc co-expression reproducibly induces between 35-58 foci in different assays (FIGS. 8A and 8B). HTLV-1 p30^{II} (HA) expression was detected in transformed colonies by immunofluorescence-microscopy (FIG. 8C); and the p30^{II} protein appeared to be distributed throughout the nucleoplasm (FIG. 8D). A high-incidence of multi-nucleated giant cells were also observed in isolated HTLV-1 p30^{II} (HA)-Myc-transformed fibroblasts that were expanded in culture, consistent with HTLV-1 p30^{II}-induced polyploidy observed during BrdU-FACS analyses (FIG. 8E; compare to control cells in FIG. 8C). Expression of HTLV-1 p30^{II} (HA) in transformed fibroblasts was confirmed by immunoblotting using a monoclonal anti-HA antibody (FIG. 8E). Indeed, these findings indicate that HTLV-1 p30^{II} markedly enhances the transforming potential of Myc and may promote genomic instability resulting in polyploidy.

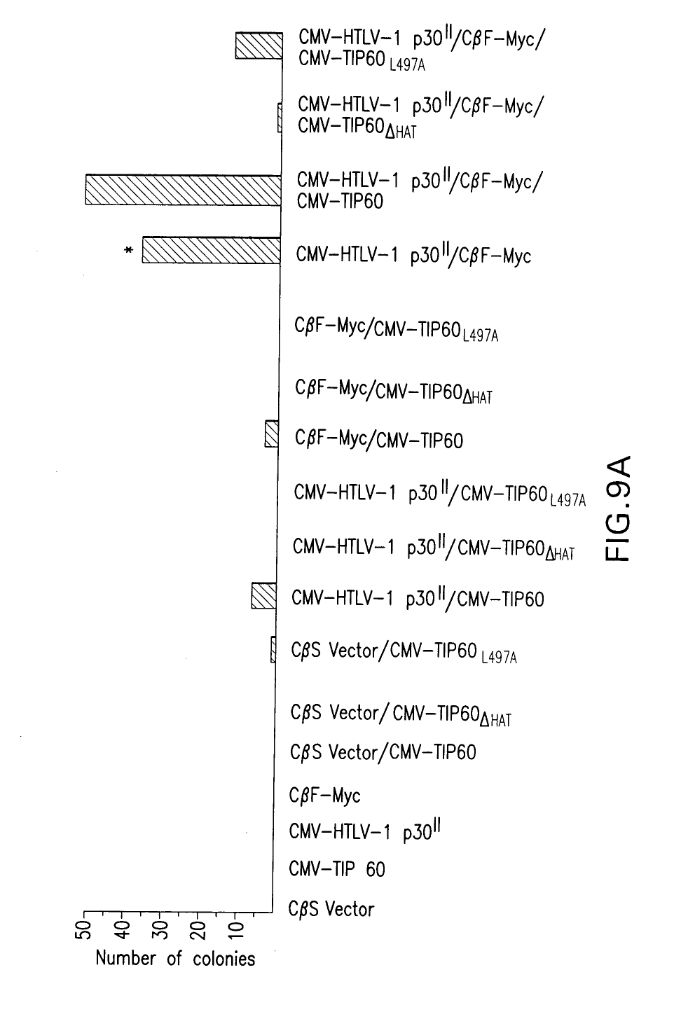

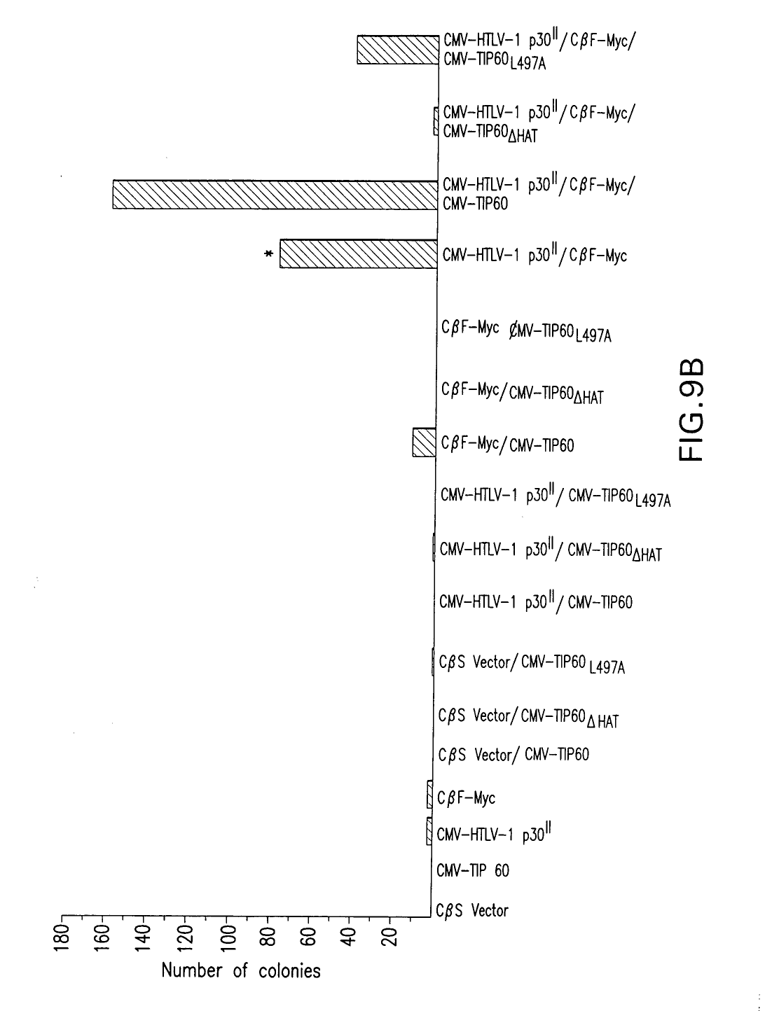

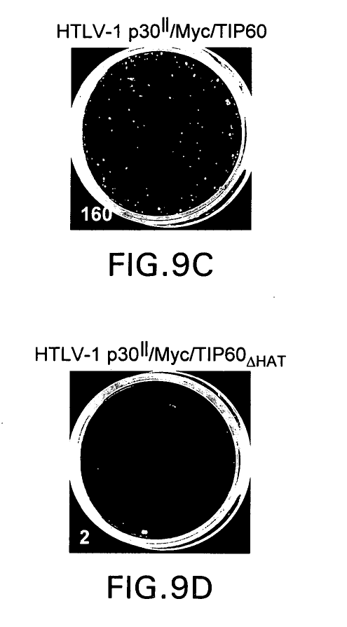

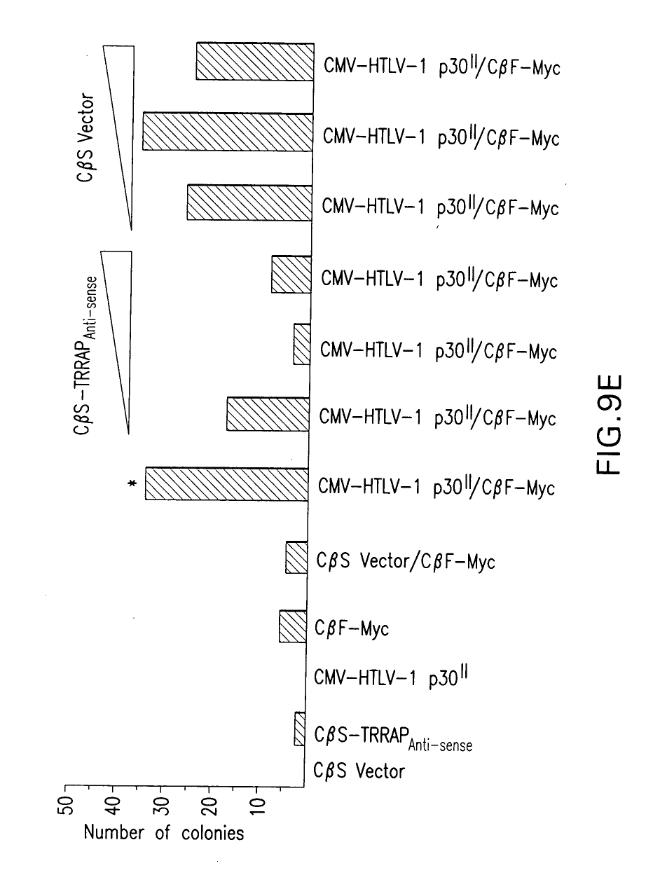

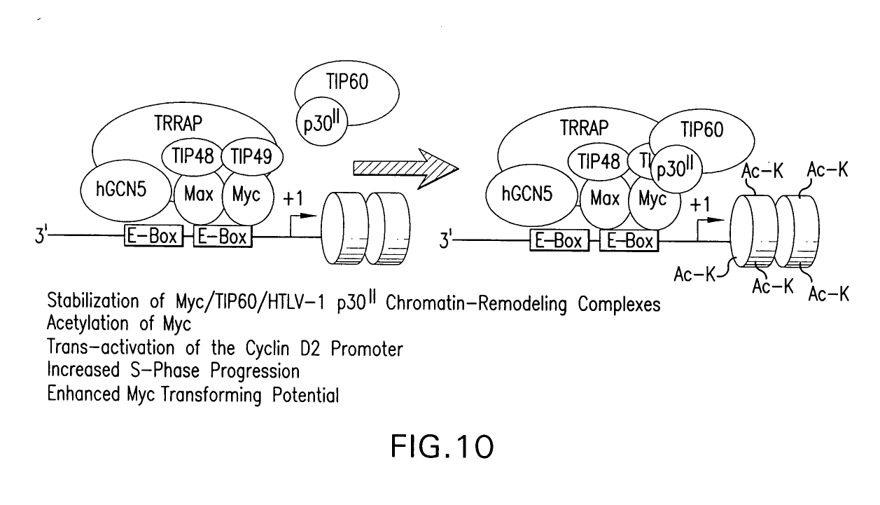

The foregoing transcriptional activation data suggested that enhancement of Myc functions by HTLV-1 p30^{II} requires the coactivators TIP60 and TRRAP/p434. Therefore, tests were performed to determine whether foci-formation induced by co-expressing HTLV-1 p30^{II} (HA)-Myc might be affected by over-expressing wild-type TIP60 or TIP60_{ΔHAT} and TIP60_{L497A} mutant proteins. Results from two independent experiments shown in FIG. 9A indicate that none of the TIP60 expression constructs, either alone or in combination with Myc, significantly induces foci-formation in immortalized human WRN^{-/-} fibroblasts. However, ectopic TIP60 markedly increases foci-formation induced by HTLV-1 p30^{II} (HA)-Myc co-expression (FIG. 9A). The trans-dominant negative TIP60_{ΔHAT} mutant completely abrogated colony formation by HTLV-1 p30^{II} (HA)-Myc, and the TIP60_{L497A} mutant partially inhibited foci-formation (FIG. 9A). Increased colony formation by HTLV-1 p30^{II} (HA)/Myc/TIP60, compared to inhibition of foci-formation by the trans-dominant-negative TIP60_{ΔHAT} mutant, is shown in FIGS. 9C and 9D. Finally, inhibition of TRRAP/p434, as a result of co-expressing increasing amounts of TRRAPₐₙₜᵢ₋ₛₑₙₛₑ RNA, also significantly decreased foci-formation by HTLV-1 p30^{II} (HA)-Myc (FIG. 9E). These findings collectively agree with the transcriptional activation data, and suggest that HTLV-1 p30^{II} enhances Myc transcriptional and transforming activities in a TIP60 HAT-and TRRAP-dependent manner (FIG. 10).

The HTLV-1 infects CD4⁺ T-cells and promotes deregulated cell-growth and lymphoproliferation associated with development of ATLL. While numerous studies have demonstrated that the viral Tax protein transcriptionally-activates growth/proliferative-signaling pathways, it has become increasingly evident that other pX-encoded regulatory factors (p12^{I}, p13^{II}, p30^{II}, Rex) are likely to perform essential functions during adult T-cell leukemogenesis. Indeed, the majority of partially-deleted HTLV-1 proviruses in ATLL patient isolates contain intact pX sequences; and alternatively-spliced ORF I and ORF II mRNAs have been detected in HTLV-1-infected transformed T-cell-lines and ATLL patient samples. Cytotoxic T-lymphocytes (CTLs) specifically targeted against ORF I and ORF II peptides have been obtained from ATLL patients suggestive that these proteins are present during in vivo HTLV-1 infections. Zhang et al. (2001) reported that p30^{II} interacts with p300/CREB-binding protein and represses Tax-mediated trans-activation from the HTLV-1 LTR (83) and differentially modulates CREB-dependent transcription (84). Nicot et al. (2004. ref. 46) and Younis et al. (2004. ref. 82) have demonstrated that p30^{II} prevents nuclear export of the doubly-spliced Tax/Rex mRNA and others have shown that p30^{II} is required for maintenance of high viral titers in a rabbit model of ATLL using an infectious HTLV-1 molecular clone, ACH.30^{II}, defective for p30^{II} production (4, 68). Interestingly, Robek et al. (1998) have previously demonstrated that p30^{II} is dispensable for immortalization and transformation of human PBMCs by ACH.p30^{II}, however, this mutant exhibited an approx 20-50% reduction in transformation-efficiency compared to the wild-type ACH.wt (60) suggesting that p30^{II} is required for the full transforming-potential of HTLV-1. The physiological role of p30^{II} in HTLV-1 pathogenesis remains unclear and it is intriguing that, similar to Tax, p30^{II}, may perform multiple functions to control viral gene expression and promote deregulation of CD4⁺ T-cell growth/proliferative pathways.

Therefore, the data presented herein demonstrates that HTLV-1 p30^{II} drastically enhances Myc-associated transcriptional and transforming activities and markedly increases S-phase progression-and polyploidy through interactions with the coactivator/HAT, TIP60 (FIG. 10). HTLV-1 p30^{II} significantly trans-activates conserved E-box enhancer elements within promoters of Myc-responsive genes, requiring TIP60 HAT activity and the transcriptional coactivator TRRAP/p434. The data presented herein indicate that, in the absence of HTLV-1 p30^{II}-interactions, ectopic TIP60 over-expression does not significantly alter Myc transcriptional and transforming activities in functional assays (see FIGS. 4A and 9A). Further, TIP60 is not detectably present in Myc-containing chromatin-remodeling complexes on the human cyclin D2 promoter, in absence of HTLV-1 p30^{II}, in Molt-4 lymphocytes (FIG. 5C). Aberrant Myc-TIP60 interactions, as a result of HTLV-1 p30^{II} or other stabilizing factors, may prominently contribute to neoplastic transformation in hematological malignancies and solid tumors where Myc functions are deregulated or that contain Myc locus mutations. Indeed, disruption of Myc-TIP60 complexes is a plausible approach for anti-cancer therapies designed to impede malignancy.

The present invention provides the first evidence, based upon biological-functional assays, that HTLV-1 p30^{II} is a novel retroviral enhancer of Myc-TIP60 transcriptional and transforming activities that likely plays an important role during adult T-cell leukemogenesis.

### EXAMPLES

### Example 1

### Plasmids, Transfections, and Cell-Culture

HeLa cells (ATCC, CCL-2) were grown in Dulbecco's Modified Eagle's Medium (D-MEM, ATCC) supplemented with 10% fetal bovine serum (FBS, Atlanta Biologicals), 100U/ml penicillin and 100 μg/ml streptomycin sulfate (Invitrogen-Life Technologies) and cultured at 37° C. under 5% CO₂. 293A Fibroblasts (Quantum Biotechnology) were cultured in ATCC 46-X medium supplemented with sodium bicarbonate (Invitrogen-Life Technologies), 10% FBS, and 100U/ml penicillin and 100 g/ml streptomycin-sulfate. Molt-4 (ATCC, CRL-1582), Jurkat E6.1 (ATCC, TIB-152) and HTLV-1-infected MJ[G11] (ATCC, CRL-8294) and HuT-102 lymphocytes (ATCC, TIB-162) were grown in RPMI medium (ATCC) supplemented with 20% FBS, 100U/ml penicillin, 100 μg/ml streptomycin-sulfate, and 20 μg/ml gentamicin-sulfate (SIGMA Chemical Corp.) and cultured under 10% CO₂. Primary HTLV-1-infected lymphocytes were obtained, after informed consent from three ATLL patients (ATL-1, ATL-2, ATL-3), and were cultured in RPMI medium supplemented with 20% FBS, 50U/ml hIL-2 (Invitrogen-Life Technologies), 100U/ml penicillin, 100 μg/ml streptomycin-sulfate, and 20 μg/ml gentamicin-sulfate. The CMV-HTLV-1 p30^{II} (HA) expression construct was kindly provided by Dr. G. Franchini (NCI, NIH) and has been reported in Koralnik et al. (1993, J. Virol. 67:2360-2366). In order to generate the human cyclin D2 promoter-luciferase reporter construct, sequences encompassing the human cyclin D2 promoter were located in GeneBank accession number U47284 clone; according to these sequences a PCR product was generated that contains 1622 nucleotides upstream of the ATG start codon. Two closely-spaced E-boxes (5′-CACGTG) are localized within the promoter region which bind Myc/Max/Mad network components (2001, Genes Dev. 15:2042-2047). This fragment was cloned into the pGL3-luciferase vector. Both E-box sequences were mutated to 5′-CTCGAG using the quick change method. The M4-tk-luciferase reporter plasmid was reported (2001, Genes Dev. 15:2042-2047; 1998, Cell 93:81-91). The CβF-FLAG-Myc, CβF-FLAG-TRRAP₁₂₆₁₋₁₅₇₉, CβS-TRRAPₐₙₜᵢ₋ₛₑₙₛₑ, and CβS constructs were described in McMahon et al. (1998, Cell 94:363-374). The pOZ-wildtype-TIP60 and pOZ-TIP60sAT expression constructs were reported in Ikura et al. (2000, Cell 102:463-473); and the CMV-TIP60_{L497A} expression plasmid was reported in Gaughan et al. (2001, J. Biol. Chem. 276:46841-46848). All transfections were performed using Lipofectamine (Invitrogen-Life Technologies) or Superfect (Qiagen) reagents as recommended.

### Example 2

### Cell-Cycle and FACS Analyses

Molt4 and Jurkat E6.1 lymphocytes were seeded in 100 mm² tissue-culture dishes and transfected with CMV-HTLV-1 p30^{II} (HA) or an empty CβS vector. Following 48 hr, cultures were split and either labeled for 4 hr by adding BrdU (BD-Pharmingen) to the medium or immediately stained using annexin-V-(FITC)/propidium iodide (BD-Pharmingen). For cell-cycle analyses, transfected BrdU-labeled cells were permeabilized and stained with a FITC-conjugated anti-BrdU antibody; and total genomic DNA was stained using 7-AAD (BD-Pharmingen). Flow cytometry was performed and data were analyzed using ModFit LT 3.0 software.

### Example 3

### Foci-Formation/Transformation Assays

Immortalized Werner's Syndrome (WRN^{-/-}) fibroblasts (2000, Nucleic Acids Res. 28:648-654) were seeded at 6×10⁵ cells in 60 mm² tissue-culture dishes in D-MEM supplemented with 10% FBS and cultured at 37° C. under 5% CO₂. Cells were transfected with an empty CβS vector, CMV-HTLV-1 p30^{II} (HA), CβF-FLAG-Myc, and combinations of CMV-HTLV-1 p30^{II} (HA)/CβF-FLAG-Myc or CβS/CβF-FLAG-Myc using Superfect reagent. Foci were observed within 2 weeks and quantified by direct counting. Expression of HTLV-1 p30^{II} (HA) was detected by fixing plates with 0.2% gluteraldehyde, 1% formaldehyde in PBS and immuno-staining using a monoclonal antibody against the HA-epitope tag (CA5, Roche Molecular Biochemicals), diluted 1:1000 in BLOTTO buffer (50 mM Tris-HCl, pH 8.0, 2 mM CaCl₂, 80 mM NaCl, 0.2% v/v NP-40, 0.02% w/v sodium azide, 5% w/v non-fat dry milk). HTLV-1 p30^{II} (HA) was visualized by immunofluorescence-microscopy. Six p30^{II}-expressing fibroblast colonies were isolated and expanded in 6-well tissue-culture plates in D-MEM supplemented with 10% FBS, 100U penicillin, and 100 mg/ml streptomycin-sulfate.

### Example 4

### Immunoprecipitations and ChIPs

Myc-interacting complexes were immunoprecipitated from transfected Jurkat E6.1 or HTLV-1-infected MJ[G11] and HuT-102 lymphocytes expressing HTLV-1 p30^{II} (HA) using a monoclonal anti-HA tag antibody. Immunoprecipitation of endogenous p30^{II}, from cultured HTLV-1-infected ATLL patient-derived lymphocytes was performed using a rabbit polyclonal antibody against the COOH-terminus of p30^{II} (anti-HTLV-1 p30^{II} antibody was generously provided by Dr. G. Franchini, NCI, NIH):(J. Virol. 67:2360-2366). Briefly, 3×10⁶ cells were harvested by centrifugation and lysed in RIPA buffer (1× PBS, 1% (v/v) IGEPAL CA-630, 0.5% sodium deoxycholate, 0.1% SDS) containing protease inhibitors: bestatin, pepstatin, antipain-dihydrochloride, chymostatin, leupeptin (50 ng/ml each. Roche Molecular Biochemicals) followed by passage through a 27.5-gauge tuberculin syringe. Immunoprecipitations were carried-out by incubating pre-cleared extracts with primary antibodies. Ten microliters of recombinant protein G-agarose (Invitrogen-Life Technologies) were added and reactions were incubated with agitation at 4° C. overnight. Matrices were pelleted by centrifugation at 6500 rpm for 5 min and washed twice with RIPA buffer. Samples were resuspended in 40 μl 2× SDS-PAGE loading buffer and bound proteins were resolved by electrophoresis through 4-15% gradient or 12.5% Tris-glycine SDS-polyacrylamide gels. Chromatin-immunoprecipitations were performed using a kit from Upstate Biotechnology. Nucleoprotein complexes were cross-linked in vivo by adding 270 μl formaldehyde to approximately 3×10⁶ Molt-4 or HTLV-1-infected MJ[G11] and HuT-102 lymphocytes in 100 mm² tissue-culture dishes for 10 min. Cells were pelleted by centrifugation and resuspended in 200 μl SDS lysis buffer. Chromatin DNA was fragmented by sonication and oligonucleosomal-protein complexes were immuno-precipitated using primary antibodies and 60 μl salmon sperm DNA/protein A agarose. Precipitated oligonucleosomal-protein complexes were washed, cross-links were reversed, and bound DNA fragments were amplified by PCR using specific oligonucleotide primer pairs:

| | | |
|---|---|---|
| PRM, | 5′ -CCCCTTCCTCCTGGAGTGAAATAC-3′; | (SEQ ID NO:1) |
| and | | |
| | 5′ -CGTGCTCTAACGCATCCTTGAGTC-3′ | (SEQ ID NO:2) |

that flank conserved E-box elements within the human cyclin D2 gene promoter or anneal within an untranslated region, UTR, 5′-ATCAGACCCTATTCTCGGCTCAGG-3′ (SEQ ID NO:3) and 5′-CAGTCAGTAAGGCACTTTATTTCCCC-3′ (SEQ ID NO:4) as described in Vervoorts et al. (2003, EMBO Rep. 4:484-490).

PCR products were electrophoresed through a 2% TAE agarose gel and visualized by ethidium bromide-staining.

Documents Cited

All sequences, patents, patent applications or other published documents cited anywhere in this specification are herein incorporated in their entirety by reference to the same extent as if each individual sequence, publication, patent, patent application or other published document was specifically and individually indicated to be incorporated by reference.

1. A method of interfering with Myc-TIP60 interaction in a cell, comprising:
contacting the cell with a nucleic acid, polypeptide, or organic molecule that inhibits Myc-TIP60 interaction in an amount and for a time sufficient to interfere with Myc-TIP60 interaction.

2. The method of claim 1, wherein the polypeptide comprises a TIP60_{ΔHAT} protein.

3. The method of claim 1, wherein the nucleic acid encodes a polypeptide comprising a TIP60_{ΔHAT} protein.

4. A method of identifying a molecule that inhibits neoplastic transformation of a cell, comprising:
contacting a test cell with a test molecule;
measuring cellular foci formed in the presence of the test molecule; and
comparing the number of foci formed by a test cell in the presence of the test molecule with the number of foci formed by a test cell in the absence of the test molecule,
wherein formation of fewer foci in the presence of the test molecule than in the absence of the test molecule indicates inhibition of neoplastic transformation, and
wherein the test cell comprises:
a first nucleic acid comprising a first expression control sequence operatively linked to a nucleotide sequence encoding the Myc transcription factor;
a second nucleic acid comprising a second expression control sequence operatively linked to a nucleotide sequence encoding human T-cell lymphotropic virus type-1 (HTLV-1) p30^{II}; and
a third nucleic acid comprising a third expression control sequence operatively linked to a nucleotide sequence encoding human TIP60.

5. The method of claim 4, wherein the second expression control sequence comprises a cytomegalovirus promoter.

6. The method of claim 4, wherein the third expression control sequence comprises a cytomegalovirus promoter.

7. The method of claim 4, wherein foci are quantitated within two weeks of exposure to the test molecule.

8. A method of identifying a molecule that interferes with Myc-TIP60 interaction, comprising:
contacting a test cell with a test molecule wherein the test cell comprises:
a first nucleic acid comprising a first expression control sequence having at least one E-box enhancer element operatively linked to a reporter gene, wherein the expression control sequence is operatively linked to the reporter gene;
a second nucleic acid comprising a second expression control sequence operatively linked to a nucleotide sequence encoding human T-cell lymphotropic virus type-1 (HTLV-1) p30^{II}; and
a third nucleic acid comprising a third expression control sequence operatively linked to a nucleotide sequence encoding human TIP60,
detecting reporter gene expression in the presence of the test molecule; and
comparing reporter gene expression in the presence of the test molecule with reporter gene expression in the absence of the test molecule,
wherein reduced reporter gene expression in the presence of the test molecule relative to reporter gene expression in the absence of the test molecule indicates inhibition of Myc-TIP60 interaction.

9. The method of claim 8, wherein the first expression control sequence comprises a promoter selected from the group consisting of a human cyclin D2 promoter and a minimal thymidine kinase promoter.

10. The method of claim 8, wherein the second expression control sequence comprises a cytomegalovirus promoter.

11. The method of claim 8, wherein the third expression control sequence comprises a cytomegalovirus promoter.

12. The method of claim 8, wherein the reporter gene encodes a protein selected from the group consisting of β-galactosidase, β-glucuronidase, an autofluorescent protein, glutathione-S-transferase, luciferase, horseradish peroxidase, and chloramphenicol acetyltransferase.

13. A method of detecting cancer in a test tissue sample, comprising:
detecting the number of Myc-TIP60 complexes in the test tissue sample; and
comparing the number of Myc-TIP60 complexes in the tissue sample with Myc-TIP60 complexes in a corresponding non-cancerous tissue,
wherein an elevated number of Myc-TIP60 complexes in the test tissue sample relative to the non-cancerous tissue indicates the presence of cancer.

14. The method of claim 13, wherein detecting Myc-TIP60 complexes comprises:
lysing cells of the test tissue sample;
forming a clear extract; and
immunoprecipitating Myc-interacting complexes with an anti-HA tag antibody.

15. The method of claim 13, wherein the test tissue sample is derived from a tissue biopsy.


SEQUENCE LISTING (4 sequences)

SEQ ID NO: 1 - DNA, 24 bp, Artificial Sequence
  PRM oligonucleotide primer
ccccttcctcctggagtgaaatac

SEQ ID NO: 2 - DNA, 24 bp, Artificial Sequence
  PRM oligonucleotide primer
cgtgctctaacgcatccttgagtc

SEQ ID NO: 3 - DNA, 24 bp, Artificial Sequence
  UTR oligonucleotide primer
atcagaccctattctcggctcagg

SEQ ID NO: 4 - DNA, 26 bp, Artificial Sequence
  UTR oligonucleotide primer
cagtcagtaaggcactttatttcccc